SEQUENCE LISTING (167 sequences)

SEQ ID NO: 1 - DNA, 20 bp, Artificial
  Primer SKY1 left
ggacatttggctgcagagat

SEQ ID NO: 2 - DNA, 20 bp, Artificial
  Primer SKY1 right
tggcaatgcactctcatcat

SEQ ID NO: 3 - DNA, 20 bp, Artificial
  Primer SKY2 left
tcaggacagacaggctgcta

SEQ ID NO: 4 - DNA, 20 bp, Artificial
  Primer SKY2 right
cctgccactgagctccttac

SEQ ID NO: 5 - DNA, 20 bp, Artificial
  Primer SKY3 left
ttctccctcatcttccaagc

SEQ ID NO: 6 - DNA, 20 bp, Artificial
  Primer SKY3 right
gcttccatccattagcaagg

SEQ ID NO: 7 - DNA, 22 bp, Artificial
  Primer SKY4 right
cctttcattccattctcttcca

SEQ ID NO: 8 - DNA, 19 bp, Artificial
  Primer SKY4 right
cgcactttatggactgcaa

SEQ ID NO: 9 - DNA, 20 bp, Artificial
  Primer SKY5 left
ccctcgtccatttcttttga

SEQ ID NO: 10 - DNA, 20 bp, Artificial
  Primer SKY5 right
cctcgaatttaatggattgc

SEQ ID NO: 11 - DNA, 21 bp, Artificial
  Primer SKY6 left
tcaatggatgcacagtgtggc

SEQ ID NO: 12 - DNA, 20 bp, Artificial
  Primer SKY6 right
tccactgaattccattgcac

SEQ ID NO: 13 - DNA, 20 bp, Artificial
  Primer SKY7 left
gggagtgcaaagggaaagat

SEQ ID NO: 14 - DNA, 20 bp, Artificial
  Primer SKY7 right
ctttccatggggtgacattc

SEQ ID NO: 15 - DNA, 22 bp, Artificial
  Primer SKY8 left
ccattcattcgagttcattacg

SEQ ID NO: 16 - DNA, 20 bp, Artificial
  Primer SKY8 right
attggaatggaatcggacag

SEQ ID NO: 17 - DNA, 20 bp, Artificial
  Primer SKY9 left
ggccgatggtcaaactgtta

SEQ ID NO: 18 - DNA, 20 bp, Artificial
  Primer SKY9 right
gaaacgggctctgaaattct

SEQ ID NO: 19 - DNA, 20 bp, Artificial
  Primer SKY10 left
ataaggggcaggtttgtcac

SEQ ID NO: 20 - DNA, 27 bp, Artificial
  Primer SKY10 right
gctacttattcagtgtttaactgacac

SEQ ID NO: 21 - DNA, 20 bp, Artificial
  Primer SKY11 left
aaagtgggtgaaggacatgg

SEQ ID NO: 22 - DNA, 19 bp, Artificial
  Primer SKY11 right
tttttgtttgtggcaggtg

SEQ ID NO: 23 - DNA, 21 bp, Artificial
  Primer SKY12 left
ttgagtcactggggataactg

SEQ ID NO: 24 - DNA, 20 bp, Artificial
  Primer SKY12 right
tatggcccacaatcacttca

SEQ ID NO: 25 - DNA, 22 bp, Artificial
  Primer SKY13 left
ggcagcctagaaagtcttgttc

SEQ ID NO: 26 - DNA, 20 bp, Artificial
  Primer SKY13 right
cccttgggattttgtctgtt

SEQ ID NO: 27 - DNA, 20 bp, Artificial
  Primer A72 forward
atttccaccgaaacccattt

SEQ ID NO: 28 - DNA, 20 bp, Artificial
  Primer A72 reverse
ctcccctaccaccaacacac

SEQ ID NO: 29 - DNA, 20 bp, Artificial
  Primer A92 forward
agggccctcacatgattaaa

SEQ ID NO: 30 - DNA, 20 bp, Artificial
  Primer A92 reverse
gcgacaccatttctttccat

SEQ ID NO: 31 - DNA, 20 bp, Artificial
  Primer A111 forward
gacatcgtggtgtctgttgc

SEQ ID NO: 32 - DNA, 20 bp, Artificial
  Primer A111 reverse
cagacgttgttcaggtcgtg

SEQ ID NO: 33 - DNA, 19 bp, Artificial
  Primer A134 forward
gcaccattagtgcgcttgt

SEQ ID NO: 34 - DNA, 21 bp, Artificial
  Primer A134 reverse
ttctccctttaccccaaattc

SEQ ID NO: 35 - DNA, 20 bp, Artificial
  Primer A158 forward
ccagcaggagtcttggagtc

SEQ ID NO: 36 - DNA, 21 bp, Artificial
  Primer A158 reverse
tgagaggcacctacggttaga

SEQ ID NO: 37 - DNA, 20 bp, Artificial
  Primer B17 forward
ccaagcatgccttcctaaag

SEQ ID NO: 38 - DNA, 20 bp, Artificial
  Primer B17 reverse
tgccttctcatctgcttgtg

SEQ ID NO: 39 - DNA, 20 bp, Artificial
  Primer B37 forward
atcctgggagatgcatcaga

SEQ ID NO: 40 - DNA, 24 bp, Artificial
  Primer B37 reverse
tgagtcctaaaccgtacacataca

SEQ ID NO: 41 - DNA, 20 bp, Artificial
  Primer B59 forward
caatggaaatgttgcaggtg

SEQ ID NO: 42 - DNA, 20 bp, Artificial
  Primer B59 reverse
tcctgccctgctgttagagt

SEQ ID NO: 43 - DNA, 20 bp, Artificial
  Primer B82 forward
gcaagggtgttgcaagttta

SEQ ID NO: 44 - DNA, 20 bp, Artificial
  Primer B82 reverse
tgcatattgtccacacatgg

SEQ ID NO: 45 - DNA, 20 bp, Artificial
  Primer B102 forward
aaagagaagggccctgtgat

SEQ ID NO: 46 - DNA, 20 bp, Artificial
  Primer B102 reverse
ctaggcaacagcactggaaa

SEQ ID NO: 47 - DNA, 19 bp, Artificial
  Primer C17 forward
aaaatccacttccccagtg

SEQ ID NO: 48 - DNA, 20 bp, Artificial
  Primer C17 reverse
gcaagaatctgggctctcac

SEQ ID NO: 49 - DNA, 20 bp, Artificial
  Primer C37 forward
cactggggaaggctgtgata

SEQ ID NO: 50 - DNA, 20 bp, Artificial
  Primer C37 reverse
cattgtcatcactgccaggt

SEQ ID NO: 51 - DNA, 21 bp, Artificial
  Primer C56 forward
cccacttcttctccaaagtcc

SEQ ID NO: 52 - DNA, 20 bp, Artificial
  Primer C56 reverse
gcacccgttttcctgatcta

SEQ ID NO: 53 - DNA, 21 bp, Artificial
  Primer C77 forward
ggggcatattctacacaccaa

SEQ ID NO: 54 - DNA, 21 bp, Artificial
  Primer C77 reverse
tgaaatggcaaacctttcaga

SEQ ID NO: 55 - DNA, 22 bp, Artificial
  Primer C97 forward
aagaatggaaggatctccaaga

SEQ ID NO: 56 - DNA, 22 bp, Artificial
  Primer C97 reverse
tctgtgcagaaatgatggattc

SEQ ID NO: 57 - DNA, 20 bp, Artificial
  Primer C120 forward
tggtagtgggaaactgctca

SEQ ID NO: 58 - DNA, 20 bp, Artificial
  Primer C120 reverse
tggtgtgctaagtggctgtc

SEQ ID NO: 59 - DNA, 23 bp, Artificial
  Primer C142 forward
gctgcagttagctaaaccaagac

SEQ ID NO: 60 - DNA, 20 bp, Artificial
  Primer C142 reverse
attctgcctgaacctccaga

SEQ ID NO: 61 - DNA, 61 bp, Artificial
  Exon trap clone et_a_001 reverse
ggtctttggctcaactcaggttccctctacctgaaatgatccaccttcagagaattggatg

SEQ ID NO: 62 - DNA, 51 bp, Artificial
  Exon trap clone et_a_002 exon 1 reverse
ctgtcttgcctcctcgatgggaaaagaaacaagcgcactaatggtgcattt

SEQ ID NO: 63 - DNA, 131 bp, Artificial
  Exon trap clone et_a_002 exon 2 reverse
ctggagcatcaggggtgtcttctatgatcaaggaaggaagccactcagggtgatagagctgcagacttctgcttggtcactctgatagctctgggaacactgtgcacctctctggctgtgatggggaaact

SEQ ID NO: 64 - DNA, 44 bp, Artificial
  Exon trap clone et_a_003 forward
cttttacatagaatggtaactccttttgcacctcgtgttttttc

SEQ ID NO: 65 - DNA, 170 bp, Artificial
  Exon trap clone et_a_004 reverse
aaagttggtagttcgctcccgggctgatgctcagagtgtggaacttgaggagctgcggtgacatcctgcagccacacgggaggtggctcctcaggggcgattgctggctgtgtcaccaccaggggacaccgggcacagcttgaagcttggggacagggagctgagaggac

SEQ ID NO: 66 - DNA, 93 bp, Artificial
  Exon trap clone et_c_001 forward
gattacatggactactatatttaaaattccttctaaactttttcccatttctgctcaattttcattctccaatatttgcaaaacttaaagttc

SEQ ID NO: 67 - DNA, 60 bp, Artificial
  Exon trap clone et_c_002 forward
gctgaacattatttctttattccagattagaggactaggattcatgggattatgcatcaa

SEQ ID NO: 68 - DNA, 68 bp, Artificial
  Exon trap clone et_c_003 reverse
ggaaatcttgaaatggcaaacctttcagaagagatggcagagactctcctacatattctgttctcaat

SEQ ID NO: 69 - DNA, 92 bp, Artificial
  Exon trap clone et_c_004 exon 1 reverse
acactggaagaattggtgtctaggcagtctgggataatagcctagttctaaggacattatcattgatccctttataggccatagacctccat

SEQ ID NO: 70 - DNA, 96 bp, Artificial
  Exon trap clone et_c_004 exon 2 reverse
ttcttcctgttggtgcaggagggtgattaagggcttttcctaccttaagttgatcaaagtggtattttcataagattaatctggcagcagaatgca

SEQ ID NO: 71 - DNA, 79 bp, Artificial
  Exon trap clone et_c_005 forward
cttggttgggaaaatatggccaccatattgctgggaaagccaccaagagtggactgttaccaatatccaagggacatga

SEQ ID NO: 72 - DNA, 20 bp, Artificial
  Primer a_r_001 forward
gcttggaacttgaggtgctc

SEQ ID NO: 73 - DNA, 20 bp, Artificial
  Primer a_r_001 reverse
ggagatgtgggcttgtgagt

SEQ ID NO: 74 - DNA, 20 bp, Artificial
  Primer a_r_002 forward
ctgtgggtgcattaggtgtg

SEQ ID NO: 75 - DNA, 19 bp, Artificial
  Primer a_r_002 reverse
ctggtacatgctgcctgct

SEQ ID NO: 76 - DNA, 23 bp, Artificial
  Primer b_f_001 forward
gacctcttttgagaaagtcagca

SEQ ID NO: 77 - DNA, 20 bp, Artificial
  Primer b_f_001 reverse
aaagcaatggcaacaaaagc

SEQ ID NO: 78 - DNA, 20 bp, Artificial
  Primer b_r_001 forward
agagggaggaaagagccatc

SEQ ID NO: 79 - DNA, 20 bp, Artificial
  Primer b_r_001 reverse
gttgtacgggctgcagaatc

SEQ ID NO: 80 - DNA, 24 bp, Artificial
  Primer b_r_002 forward
tgagtcctaaaccgtacacataca

SEQ ID NO: 81 - DNA, 20 bp, Artificial
  Primer b_r_002 reverse
tttctgtgcgtgagaacaca

SEQ ID NO: 82 - DNA, 20 bp, Artificial
  Primer c_f_001 forward
tctctgtggtgctgatcctg

SEQ ID NO: 83 - DNA, 20 bp, Artificial
  Primer c_f_001 reverse
gcaagaatctgggctctcac

SEQ ID NO: 84 - DNA, 20 bp, Artificial
  Primer c_f_001b forward
atccctattcgccccttaga

SEQ ID NO: 85 - DNA, 20 bp, Artificial
  Primer c_f_002 forward
acctcagggtgcagctttta

SEQ ID NO: 86 - DNA, 20 bp, Artificial
  Primer c_f_002 reverse
tgagcagtttcccactacca

SEQ ID NO: 87 - DNA, 23 bp, Artificial
  Primer c_f_003 forward
gctgcagttagctaaaccaagac

SEQ ID NO: 88 - DNA, 20 bp, Artificial
  Primer c_f_003 reverse
ttctgcaagggtctggttct

SEQ ID NO: 89 - DNA, 18 bp, Artificial
  Primer c_r_001 forward
cacagaagccagggatcg

SEQ ID NO: 90 - DNA, 18 bp, Artificial
  Primer c_r_001 reverse
gcatctcgccctttcctc

SEQ ID NO: 91 - DNA, 20 bp, Artificial
  Primer c_r_002 forward
caacactgtacaccgcaaca

SEQ ID NO: 92 - DNA, 21 bp, Artificial
  Primer c_r_002 reverse
ttctccaaagtccgatacctg

SEQ ID NO: 93 - DNA, 21 bp, Artificial
  Primer c_r_003 forward
tggagacattcacaacgtcaa

SEQ ID NO: 94 - DNA, 20 bp, Artificial
  Primer c_r_003 reverse
tggtagtgggaaactgctca

SEQ ID NO: 95 - DNA, 20 bp, Artificial
  Primer c_r_004 forward
agctgcctgacttcttggaa

SEQ ID NO: 96 - DNA, 20 bp, Artificial
  Primer c_r_004 reverse
cttgcccacaccttgatctc

SEQ ID NO: 97 - DNA, 20 bp, Artificial
  Primer c_r_005 forward
cgtgctggattcctatttgg

SEQ ID NO: 98 - DNA, 21 bp, Artificial
  Primer c_r_005 reverse
cccacttcttctccaaagtcc

SEQ ID NO: 99 - DNA, 20 bp, Artificial
  Primer AdlYEx1 forward
gactcctggccttgacttga

SEQ ID NO: 100 - DNA, 20 bp, Artificial
  Primer cf1 forward
tctctgtggtgctgatcctg

SEQ ID NO: 101 - DNA, 23 bp, Artificial
  Primer cf1-117 forward
ggaggagcaaaaacaagaagaga

SEQ ID NO: 102 - DNA, 19 bp, Artificial
  Primer cf1-205 forward
actgatgagcacgggaacc

SEQ ID NO: 103 - DNA, 19 bp, Artificial
  Primer C17c forward
tccatcctgaaagtgcctg

SEQ ID NO: 104 - DNA, 20 bp, Artificial
  Primer C18 forward
acatgtatacatgctgccaa

SEQ ID NO: 105 - DNA, 20 bp, Artificial
  Primer AdlYEx5 forward
cagcgaaggaaagcacattt

SEQ ID NO: 106 - DNA, 18 bp, Artificial
  Primer cf1-1815 forward
ggcgacctgaaggggact

SEQ ID NO: 107 - DNA, 20 bp, Artificial
  Primer C21 forward
ctgtccagtcctcaggaagc

SEQ ID NO: 108 - DNA, 18 bp, Artificial
  Primer cf1-4679 forward
gaagcatccaccaaagcg

SEQ ID NO: 109 - DNA, 18 bp, Artificial
  Primer cf1-4a forward
acagcgggcgctatgagt

SEQ ID NO: 110 - DNA, 20 bp, Artificial
  Primer AdlYEx2 reverse
caggatcagcaccacagaga

SEQ ID NO: 111 - DNA, 21 bp, Artificial
  Primer C17b reverse
ctggggaagttggattttctc

SEQ ID NO: 112 - DNA, 18 bp, Artificial
  Primer cf1-227 reverse
accaggttcccgtgctca

SEQ ID NO: 113 - DNA, 20 bp, Artificial
  Primer cf1 reverse
gcaagaatctgggctctcac

SEQ ID NO: 114 - DNA, 20 bp, Artificial
  Primer C17c reverse
actgtgattcccaccgtgat

SEQ ID NO: 115 - DNA, 20 bp, Artificial
  Primer C18 reverse
ttgttttgaggaacgcctct

SEQ ID NO: 116 - DNA, 19 bp, Artificial
  Primer cf1-2079 reverse
ggatgtgggatctggtgag

SEQ ID NO: 117 - DNA, 21 bp, Artificial
  Primer AdlYEx5 reverse
gggtgtaattttctcccattg

SEQ ID NO: 118 - DNA, 20 bp, Artificial
  Primer cf1-4810 reverse
cgtccgtttcagcagtgaca

SEQ ID NO: 119 - DNA, 18 bp, Artificial
  Primer cf1-4b reverse
ctgacgtccgtcctctgc

SEQ ID NO: 120 - DNA, 20 bp, Artificial
  Primer cf1-6453 reverse
atggacagtgatccggtttc

SEQ ID NO: 121 - DNA, 20 bp, Artificial
  Primer cf1-6559 reverse
tgagctgcacgatcaacctc

SEQ ID NO: 122 - DNA, 20 bp, Artificial
  RT-PCR Primer AdlYEx1 forward
gactcctggccttgacttga

SEQ ID NO: 123 - DNA, 20 bp, Artificial
  RT-PCR Primer cf1 forward
tctctgtggtgctgatcctg

SEQ ID NO: 124 - DNA, 20 bp, Artificial
  RT-PCR Primer AdlYEx5 forward
cagcgaaggaaagcacattt

SEQ ID NO: 125 - DNA, 20 bp, Artificial
  RT-PCR Primer C21 forward
ctgtccagtcctcaggaagc

SEQ ID NO: 126 - DNA, 18 bp, Artificial
  cf1-4a forward
acagcgggcgctatgagt

SEQ ID NO: 127 - DNA, 20 bp, Artificial
  RT-PCR Primer AdlYEx2 reverse
caggatcagcaccacagaga

SEQ ID NO: 128 - DNA, 20 bp, Artificial
  RT-PCR Primer cf1 reverse
gcaagaatctgggctctcac

SEQ ID NO: 129 - DNA, 21 bp, Artificial
  RT-PCR Primer AdlYEx5 reverse
gggtgtaattttctcccattg

SEQ ID NO: 130 - DNA, 18 bp, Artificial
  RT-PCR Primer cf1-4b reverse
ctgacgtccgtcctctgc

SEQ ID NO: 131 - DNA, 20 bp, Artificial
  RT-PCR Primer cf1-6452 reverse
atggacagtgatccggtttc

SEQ ID NO: 132 - DNA, 19 bp, Artificial
  RT-PCR primer for exon trap clone eta2 forward
gcaccattagtgcgcttgt

SEQ ID NO: 133 - DNA, 20 bp, Artificial
  RT-PCR primer for exon trap clone eta2 reverse
gagcatcaggggtgtcttct

SEQ ID NO: 134 - DNA, 26 bp, Artificial
  RT-PCR primer for exon trap clone eta3a forward
ttacatagaatggtaactccttttgc

SEQ ID NO: 135 - DNA, 20 bp, Artificial
  RT-PCR primer for exon trap clone eta3b forward
aactccttttgcacctcgtg

SEQ ID NO: 136 - DNA, 20 bp, Artificial
  RT-PCR primer for exon trap clone eta4a reverse
gctgatgctcagagtgtgga

SEQ ID NO: 137 - DNA, 19 bp, Artificial
  RT-PCR primer for exon trap clone eta4b reverse
gattgctggctgtgtcacc

SEQ ID NO: 138 - DNA, 26 bp, Artificial
  RT-PCR primer for exon trap clone etc1a forward
tttaaaattccttctaaactttttcc

SEQ ID NO: 139 - DNA, 21 bp, Artificial
  RT-PCR primer for exon trap clone etc1b forward
cccatttctgctcaattttca

SEQ ID NO: 140 - DNA, 26 bp, Artificial
  RT-PCR primer for exon trap clone etc2a forward
gctgaacattatttctttattccaga

SEQ ID NO: 141 - DNA, 23 bp, Artificial
  RT-PCR primer for exon trap clone etc2b forward
agaggactaggattcatgggatt

SEQ ID NO: 142 - DNA, 21 bp, Artificial
  RT-PCR primer for exon trap clone etc3a reverse
tgaaatggcaaacctttcaga

SEQ ID NO: 143 - DNA, 23 bp, Artificial
  RT-PCR primer for exon trap clone etc3b reverse
ggcagagactctcctacatattc

SEQ ID NO: 144 - DNA, 21 bp, Artificial
  RT-PCR primer for exon trap clone etc4 forward
tggcctataaagggatcaatg

SEQ ID NO: 145 - DNA, 20 bp, Artificial
  RT-PCR primer for exon trap clone etc4 reverse
ggtgcaggagggtgattaag

SEQ ID NO: 146 - DNA, 20 bp, Artificial
  RT-PCR primer for exon trap clone etc5a forward
gaaagccaccaagagtggac

SEQ ID NO: 147 - DNA, 21 bp, Artificial
  RT-PCR primer for exon trap clone etc5b forward
accaatatccaagggacatga

SEQ ID NO: 148 - DNA, 10 bp, Artificial
  Exon 1 Intron/Exon ADLX boundary
gagctgcctc

SEQ ID NO: 149 - DNA, 10 bp, Artificial
  Exon 2 Intron/Exon ADLX boundary
gagctgcctc

SEQ ID NO: 150 - DNA, 20 bp, Artificial
  Exon 2 Intron/Exon ADLX boundary
tctacctcaggtatccgaga

SEQ ID NO: 151 - DNA, 20 bp, Artificial
  Exon 2 Intron/Exon ADLY boundary
tctacctcaggtatccgaga

SEQ ID NO: 152 - DNA, 20 bp, Artificial
  Exon 5 Intron/Exon ADLX boundary
tttgttttaggaattctgaa

SEQ ID NO: 153 - DNA, 20 bp, Artificial
  Exon 5 Intron/Exon ADLY boundary
tttgttttaggaattctgaa

SEQ ID NO: 154 - DNA, 20 bp, Artificial
  Exon 6 Intron/Exon ADLX boundary
ttttctccaggagctcttat

SEQ ID NO: 155 - DNA, 20 bp, Artificial
  Exon 6 Intron/Exon ADLY boundary
ttttctccaggagttcttat

SEQ ID NO: 156 - DNA, 20 bp, Artificial
  Exon 7 Intron/Exon ADLX boundary
ttttctgtagttttgatagc

SEQ ID NO: 157 - DNA, 20 bp, Artificial
  Exon 7 Intron/Exon ADLY boundary
ttttctgtagttttgatagt

SEQ ID NO: 158 - DNA, 20 bp, Artificial
  Exon 1 Exon/Intron ADLX boundary
ccaaggacaggtgaggaccc

SEQ ID NO: 159 - DNA, 20 bp, Artificial
  Exon 1 Exon/Intron ADLY boundary
ccaaggataggtgaggaccc

SEQ ID NO: 160 - DNA, 20 bp, Artificial
  Exon 2 Exon/Intron ADLX boundary
tcaatttggggtttgtacca

SEQ ID NO: 161 - DNA, 20 bp, Artificial
  Exon 2 Exon/Intron ADLY boundary
tcaatttggggtttgtacca

SEQ ID NO: 162 - DNA, 20 bp, Artificial
  Exon 5 Exon/Intron ADLX boundary
gtttccacaggtaatatgtt

SEQ ID NO: 163 - DNA, 20 bp, Artificial
  Exon 5 Exon/Intron ADLY boundary
gtttccacatgtaagatttt

SEQ ID NO: 164 - DNA, 20 bp, Artificial
  Exon 6 Exon/Intron ADLX boundary
cgctcttcaggtaggcagct

SEQ ID NO: 165 - DNA, 20 bp, Artificial
  Exon 6 Exon/Intron ADLY boundary
cgcttttcaggtaggcagct

SEQ ID NO: 166 - DNA, 11 bp, Artificial
  Exon 7 Exon/Intron ADLX boundary
atattctcccc

SEQ ID NO: 167 - DNA, 11 bp, Artificial
  Exon 7 Exon/Intron ADLY boundary
atattctcccc

(19) United States
(12) Patent Application Publication
(10) Pub. No.: US 2006/0234225 A1
(43) Pub. Date: Oct. 19, 2006

(54) Height-related gene

(76) Inventors: Gudrun A Rappold, Heidelberg (DE); Stefan Kirsch, Weinheim (DE)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 10/511,708

(22) PCT Filed: Apr. 25, 2003
(86) PCT No.: PCT/EP03/04546

§ 371 Date: Dec. 7, 2005

(30) Foreign Application Priority Data
Apr. 26, 2002 (GB) 0209640.2
Jul. 1, 2002 (GB) 0215188.4

(51) Int. Cl. C12Q 1/68; C07H 21/04

(52) U.S. Cl. 435/6; 536/23.1

(57) ABSTRACT

The application relates to isolated regions and genes from the Y chromosome which encompass the Y specific growth gene GCY. Probes and primers are also provided.

The sex-related height difference in humans is thought to be caused mainly by two components: first, a hormonal component determined by the sex dimorphism of bioactive gonadal steroids and second, a genetic component attributed to a Y-specific growth gene, termed GCY (Tanner, et al. 1966; Smith, et al. 1985; Ogata and Matsuo, 1992). Despite extensive mapping attempts for this gene on the human Y chromosome (Ogata, et al. 1995, Salo, et al. 1995, Rousseaux-Prevost, et al. 1996, De Rosa, et al. 1997), its precise position remains unknown. Recent evidence shows that inappropriate cytogenetic methodology in the characterization of Y-chromosomal terminal deletions has brought about some of the difficulties in elucidating the GCY-critical region. In order to overcome these problems, the inventors have considered only patients presenting de novo interstitial deletions for the GCY analysis on the Y chromosome (Kirsch, et al. 2000). This approach allows the assignment of GCY to a particular chromosomal interval without excluding the presence of X0-mosaicism and/or i(Yp) and idic(Yq11) chromosomes in patients with terminal deletions.

The direct comparison of overlapping interstitial deletions in seven adult males with normal height, one male with borderline height, and one patient with a large interstitial deletion and short stature resulted in the confirmation of the GCY critical interval between markers DYZ3 and DYS 11. This region roughly encompasses 1.6-1.7 Mb of genomic DNA. To improve the resolution in the region of interest close to the centromere, the inventors have established additional new STS markers specific for this part of the chromosome using our bacterial artificial chromosome (BAC)/P1-derived artificial chromosome (PAC) contig. Molecular deletion analysis using these new Y-chromosomal STSs allowed the inventors to narrow down the critical interval to a genomic region of 700 kb.

Preferably the regions are to the exclusion of the regions of chromosomes on each side of the defined regions.

Preferably the region is between SKY1 and sY83. It may include one or both the SKY1 and the sY83 regions. Preferably the region is between SKY8 and sY83 (preferably includes one or both of the SKY8 and sY83 regions), or SKY1 and SKY4.

The invention provides an isolated region of the Y chromosome between DYZ3 and DYS11 which encompasses GCY. Preferably the Y chromosome is a human Y chromosome.

The preferred region is between sY79 and sY81, preferably to the exclusion of the region of the Y chromosome outside that area of the chromosome.

Primers for use in GCY studies are also provided.

The invention further provides isolated gene/pseudogene sequences which contributes the sex related height difference in humans. These may be one or more of the gene or pseudogene sequences identified in one or more of the figures.

The invention further encompasses proteins having the same function as GCY protein and which have greater than 65% homology, greater than 70% homology, greater than 75% homology, greater than 80% homology, greater than 85% homology, preferably greater than 90% homology, and most preferably greater than 95% homology to the GCY protein. Preferably this has GCY gene activity, for example it has an effect on the height of a male mammal when expressed in that mammal.

Primers for use in detecting or amplifying a region of GCY are also provided. They may be labelled using radioactive or non-radioactive labels known in the art and used using well known methods. These methods include PCR, Southern or Northern blotting.

Experimental evidence will now be described in detail with reference to the figures in which:

Table 1 is a comparison of the adult height of patients and their siblings.

Table 2 is a table of new Y chromosomal STSs

Table 3 is the PCR/restriction digest analysis of sequence family variants in the AZFc region

Table 4 is a summary of BAC and PAC clones identified during physical map creation.

Table 5 is a summary of the genomic primers that will be used for microdeletion screening in adult males with idiopathic short stature.

Table 6 is a summary of the sequences of the isolated exon trap clones

Table 7A is a summary of primer pairs for predicted genes,
7B is a summary of primer pairs specific for the Y-copy of Adlican (ADLY),
7C is summary of RT-PCR primer sequences for ADLY,

Table 8 is RT-PCR primer sequences for exon trap clones,

Tables 9a & b are tables showing homology of exons between ADLX and ADLY.

Table 10 is a summary of sequence divergence of genes/pseudogenes from the GCY region and their homology.

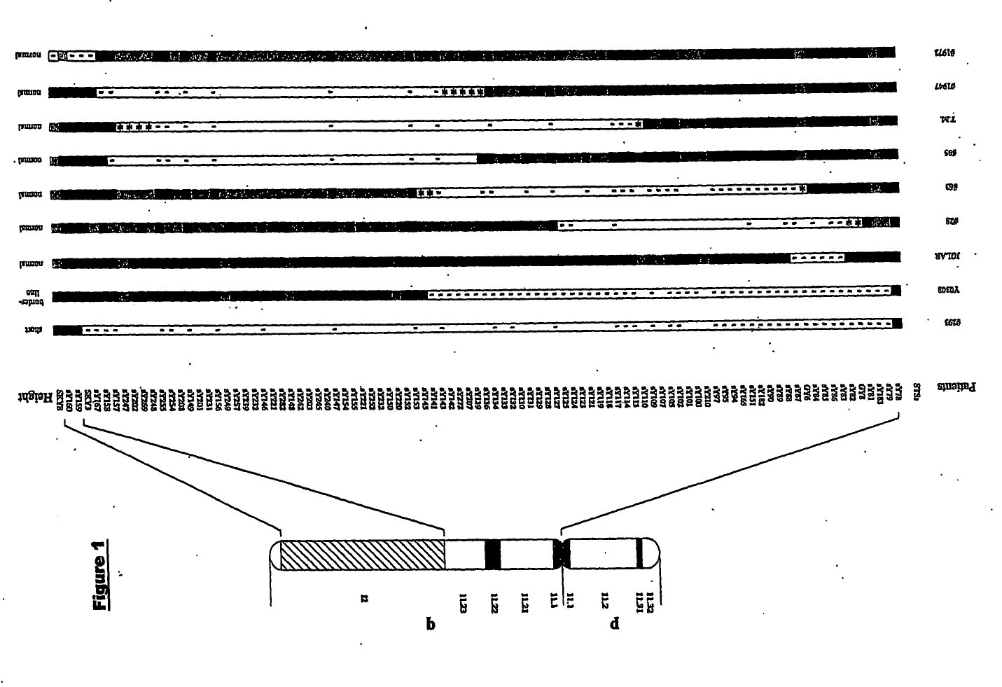

FIG. 1. Deletion mapping on the long arm of the human Y chromosome.

A diagram of the human Y chromosome with Yp telomere to the left and Yq telomere to the right is presented at the top. Shown below are the results of low-resolution analysis of Y-chromosomes of adult males with normal height or short stature. Along the top border, 95 Y-chromosomal STSs are listed. Except for SKY3 and SKY8 (see Table 2 for detail), all other STSs were previously reported (Vollrath et al., 1992, Jones et al., 1994, Reijo et al., 1995). Blank spaces or grey boxes indicate inferred absence or presence of markers for which assay was not performed. Asterisks indicate markers in the respective breakpoint regions which could not be tested. In all cases where previously published data of the patients were re-investigated, the identical DNA sample used for the primary analysis was studied. (Please note that the proximal as well as the distal breakpoint of the interstitial deletion of patient #293 resides within satellite type II sequences.)

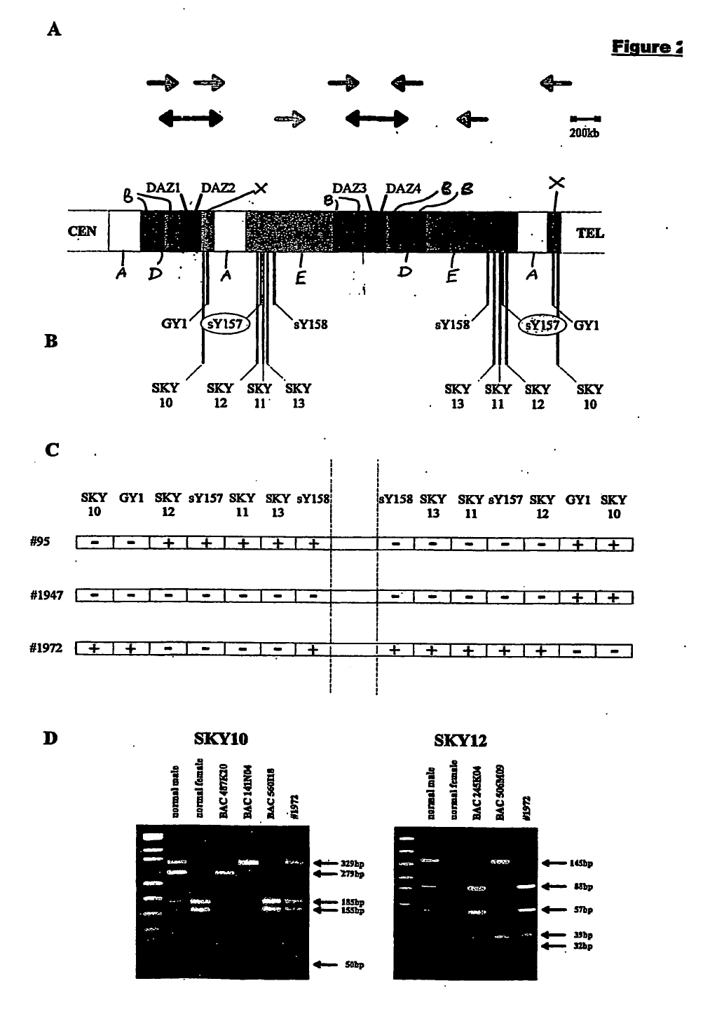

FIG. 2. Sequence family variant (SFV) typing in the human DAZ locus in distal Yq11.23.

A. Overview and amplicon structure of the human Y chromosome in the vicinity of the human DAZ cluster. Each amplicon is represented by specific bands (A, B, D, E, X). Shown above are arrows indicating the orientation of each member of an amplicon family with respect to each other. The amplicon indicated by bands X arose from a portion of chromosome 1 that was transposed to the distal end of the DAZ cluster and partially duplicated.

B. Precise position of selected Y-specific STSs and the SFVs according to the physical map of the human Y chromosome. Marker sY157 is highlighted as it was suspected to be present in only one copy by multiplex PCR analysis (see text for detail).

C. Summary of STS and SFV analysis in patients with Y-chromosomal rearrangements within the human DAZ cluster region. Grey boxes indicate inferred absence or presence of markers.

D. Sequence family variant typing of SKY10 and SKY12 in genomic DNA of patient #1972. Assay is described in Table 3. Along the right are listed fragment sizes (in bp). Products are separated by electrophoresis in 3% NuSieve agarose (3:1) and visualized by ethidium bromide staining.

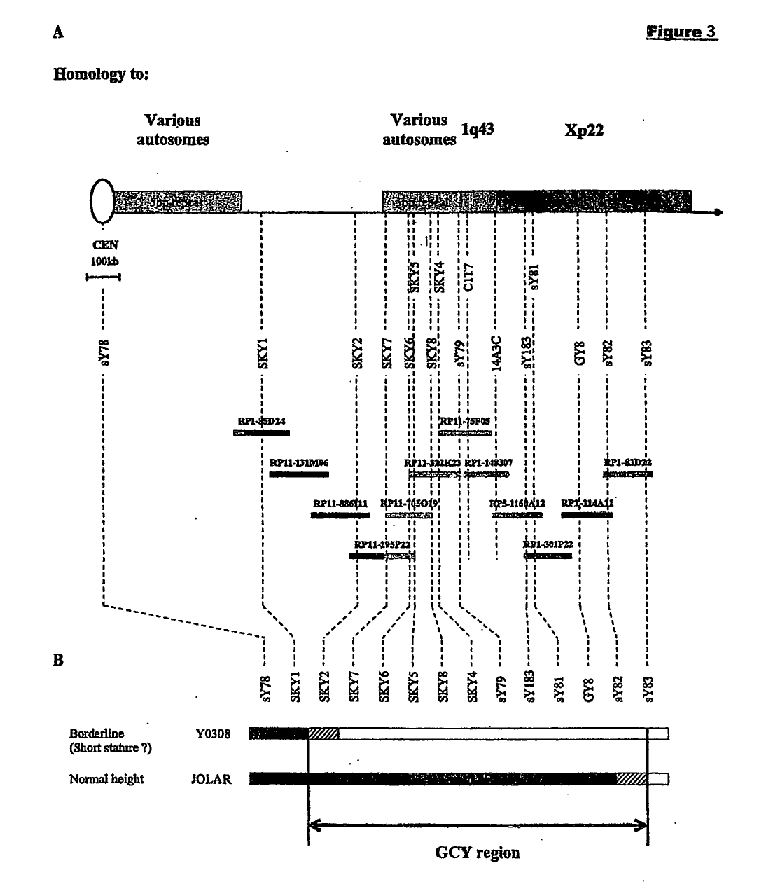

FIG. 3. Schematic representation of the organization of the long arm pericentromeric region of the human Y chromosome

A. Diagram showing the distribution of major tandem repeat blocks and general organization of sequence homologies. Basically, the region can be subdivided in three distinct intervals: a proximal region characterized by 5 bp satellite sequences (G), a central region with high homology to chromosome 1 (O), and a distal region composed of X/Y-homologous sequences (B). Below the precise position of the newly established and previously published STS markers in this region are illustrated. At the bottom border, the PAC/BAC contig constructed with the aid of the new STS markers is shown. Prefixes RP1, 5 indicate PAC clones and RP11 BAC clones, respectively.

B. Localization of the GCY critical interval as defined by high-resolution STS mapping in patients with short stature and normal height. Black boxes indicate the presence, white boxes the absence of the respective STS. Striped boxes depict the dosage unknown regions where the breakpoint resides.

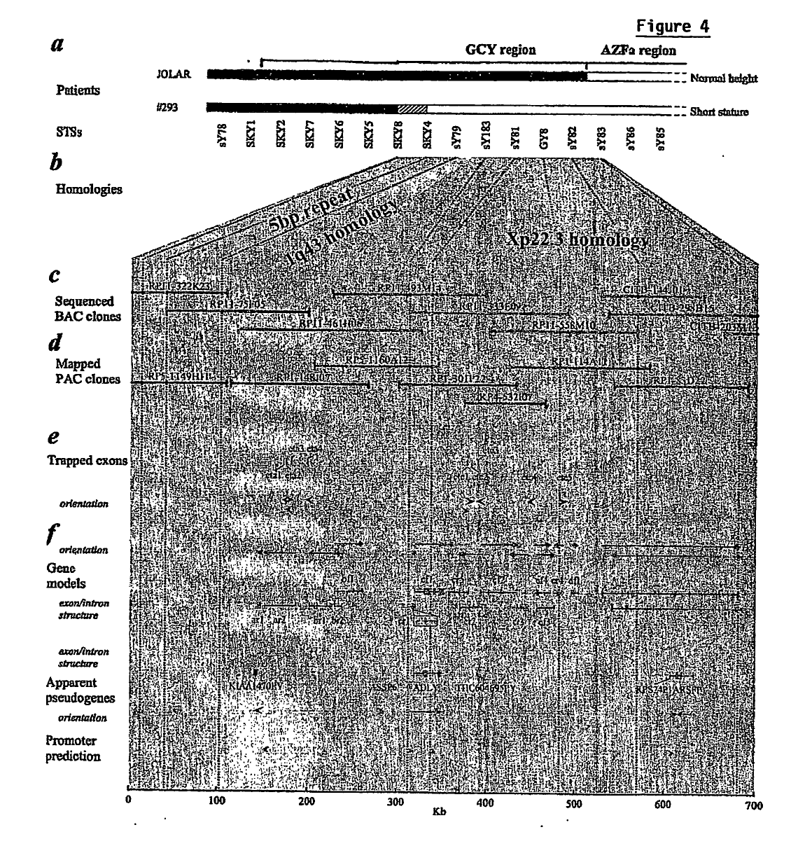

FIG. 4. Molecular characterization of the GCY critical region a. Schematic illustration of the deletions in the two most crucial patients. SKY1 and sY83 demarcate its boundaries because clone Y0308 was found to have a different deletion (see FIG. 3) marking SKY1 as one of its boundaries. The AZFa region distally adjacent to the GCY region is indicated. b. Structural compartmentalization in three segments with distinct homologies. The segment composed of 5bp repeats is shown in green, the segment homologous to chromosomal subinterval 1q43 in orange, and the segment homologous to Xp22 in blue. c. Detailed description of annotated BAC clones sourcing the genomic sequence of the GCY region. d. Precise positioning of PAC clones used as substrates for exon amplification. e. Location of all exon trap clones. Due to its small size and limited single-copy content contained within exon trap clone eta1 was not amenable for further experimental analyses f. Documentation of all in silico generated data sets in subsequent layers: gene models (orientation; exon/intron structure)—apparent pseudogenes (exon/intron structure; orientation)—promoters. Orientation of gene models can be deduced by colour (red: orientation towards the centromere; blue: orientation towards the telomere). Please note that the chromosomal region covered by CITB-144J01, CITB-298B15, and CITB-203M13 was already intensively studied in Sargent et al. 1999.

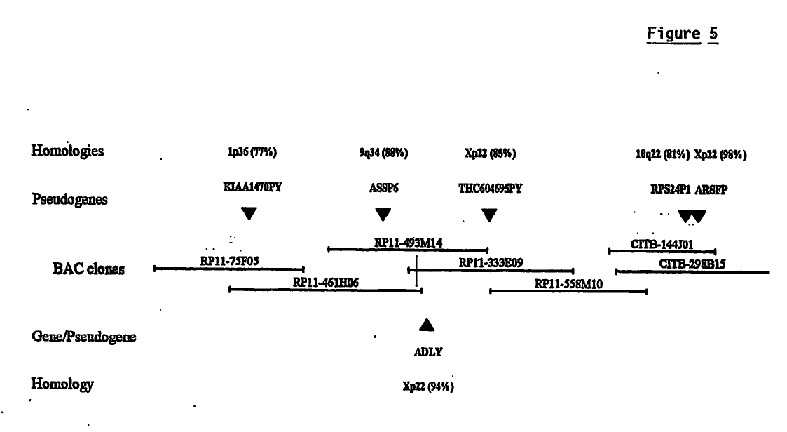

FIG. 5. Homology comparisons between genes/pseudogenes of the GCY region and their functional progenitors. Precise location of the Y-chromosomal copies is indicated. Gene pair-specific homology and subchromosomal location of the actual structural gene is shown in blue.

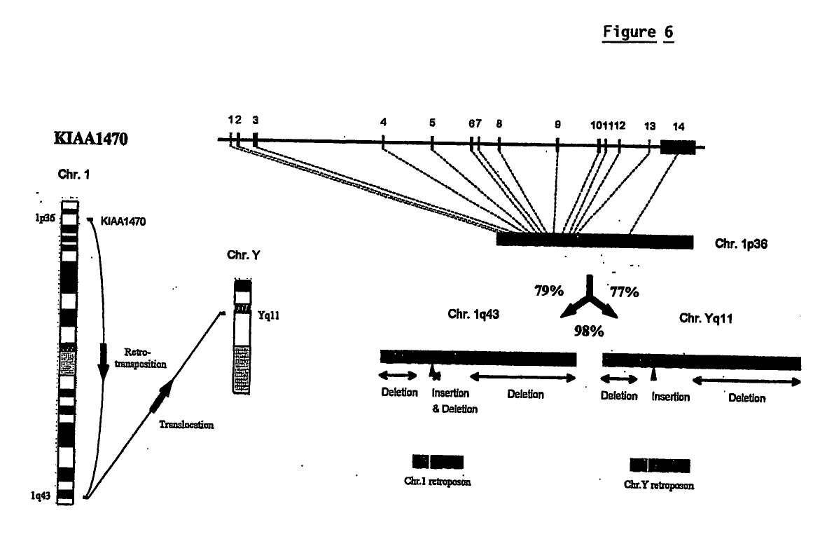

FIG. 6. Evolutionary history of KIAA1470. On the left, chromosomal movements are illustrated. The upper lateral bar shows the exon/intron structure of the functional progenitor in 1p36. Successive degenerating events have shaped KIAA1470 into the two pseudogenes on 1q43 and Yq11. Both copies on 1q43 and Yq11 share a 98% nucleotide sequence homology with each other, the highest among the 12 retroposons of the KIAA1470 gene family. They show 77% and 79% homology to the master gene.

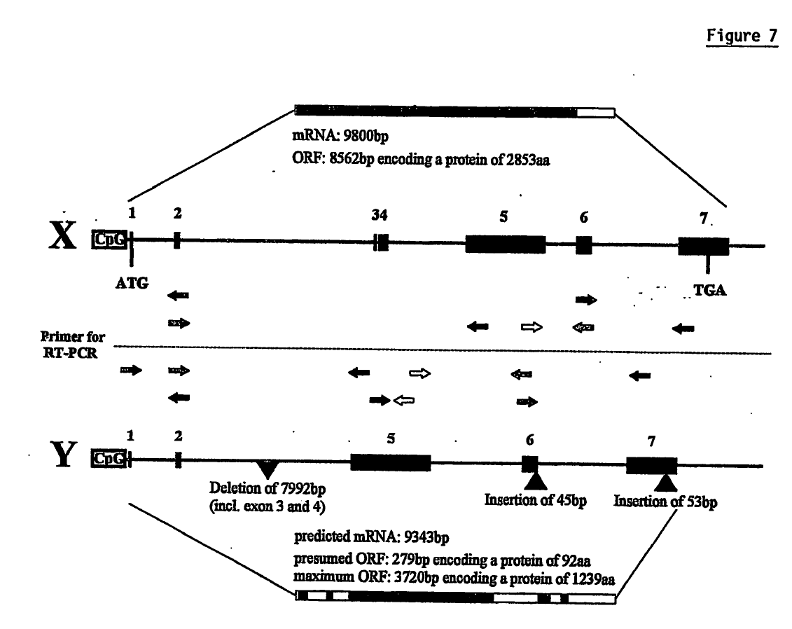

FIG. 7. Comparison of the structural features of the X- and Y-specific adlican gene/pseudogene. The coding exons of ADLX are illustrated as boxes. Corresponding putative exons of ADLY (also presented as boxes) were identified by homology searches. Major rearrangements in the putative transcriptional unit of ADLY are highlighted as black triangles. Sizes of mRNAs and ORF are presented for ADLX and ADLY. Primers used in RT-PCR assays are shown at their respective locations. Identical colouring above and below the separation line indicate non-selectivity for both transcripts. RT-PCR primers exclusively presented below the line are specific for ADLY.

Materials and Methods

Defining the GCY Critical Region

Selection of Patients

Patients #293, JOLAR, #28, #63 and #95 have been described clinically in detail elsewhere (Skare et al. 1990; Ma et al. 1993; Foresta et al. 1998; Kleiman et al. 1999). Patient Y0308 corresponds to case 1 in the study of Pryor et al. 1997. Patients T.M., #1947 and #1972 are phenotypically normal males suffering from idiopathic infertility. Genomic DNA samples were extracted from peripheral blood leukocytes (#28, #63, #95, Y0308, T.M., #1947, #1972) or from lymphoblastoid cell lines (#293, JOLAR). DNA isolated from peripheral blood leukocytes of normal males and females served as internal controls.

Height Assessment

As all individuals are of diverse ethnic origins, height was compared to the respective national height standards (Table 1). Patients were of similar age range. When possible, special attention was given to adult height comparisons between parents and siblings. Data are summarized along with the height standard deviation score (SDS) in Table 1. To calculate the SDS, mean adult height and the standard deviation were taken from the corresponding national physical growth studies.

PCR Analysis

Reactions were performed in a total volume of 50 μl (75 mM Tris/HCl pH9.0, 20 mM (NH₄)₂ SO₄, 0.1% (w/v) Tween20, 1.5 mM MgCl₂) containing 1.0 mM of each oligonucleotide primer, 100 ng genomic DNA as template, 5 units of Taq DNA polymerase (Eurogentec), and each dNTP at 1 mM in a thermocycler (MJ Research, Inc.) as follows: After an initial denaturation step of 95° C. for 5 min, samples were subjected to 30 cycles consisting of 30 sec at 94° C., 30 sec at 60° C. and 1 min at 72° C. followed by a final extension step of 5 min at 72° C. The Multiplex PCR was carried out as described in Henegariu et al. 1994 with minor modifications. Alu-Alu PCR reactions were essentially carried out as described in Nelson et al. 1991. Amplification products smaller than 1 kb were resolved on 3% NuSieve agarose/1% SeaKem GTG agarose (FMC) in 1×TBE (0.089 M Tris-borate/0.089 M boric acid/20 mM EDTA, pH 8.0). For amplification products larger than 1 kb as well as products from Alu-Alu-PCR, 1.5% SeaKem GTG agarose gels in 1×TBE were used for separation.

PCR Primers

Y-specific STSs, loci and PCR conditions have been described previously (Vollrath et al. 1992; Jones et al. 1994; Reijo et al. 1995). Sequences of new Y-chromosomal STSs are listed in Table 2. Y-specific STSs termed SKY were either derived from YAC, BAC and PAC end sequences or from clone-internal sequences amplified by various combinations of Alu primers. Primers for the markers SKY10, 11, 12, and 13 were designed to amplify fragments spanning unique restriction sites within the genomic DAZ locus (SKY10 from RP11-487K20 (AC024067), RP11-70G12 (AC006983), RP11-141N04 (AC008272), RP11-366C06 (AC015973), RP11-560118 (AC053522), RP11-175B09 (AL359453), SKY11 and SKY12 from RP11-245K04 (AC007965), RP11-100J21 (AC017005), RP11-506M09 (AC016752), RP11-589P14 (AC025246) and SKY13 from RP11-100J21 (AC017005), RP11-589P14 (AC025246), RP11-823D08 (AC073649), RP11-251M08 (AC010682), RP11-978G18 (AC073893)) in order to detect ‘sequence family variants’ (SFVs).

Restriction Analysis of PCR Products

PCR products were resolved on agarose gels, the appropriate gel bands cut out and the DNA isolated with GFX™ PCR DNA and Gel Band Purification Kit (Amersham Pharmacia Biotech, Inc.) according to the manufacturer's protocol. Fragments amplified from SKY5 and SKY6 were digested with TaqI and BsmI respectively. To detect SFVs at SKY10, SKY11, SKY12 and SKY13, PCR products were digested with restriction enzymes as listed in Table 3.

Sequencing of BAC/PAC/YAC end Fragments

DNA from BAC/PAC clones selected for end sequencing were purified with the Nucleobond PC100 Kit Macherey-Nagel) according to the manufacturer's instructions. End fragments were directly sequenced using the Thermosequenase Fluorescent Labelled Primer Cycle Sequencing Kit (Pharmacia) and analyzed on a Pharmacia A.L.F. express (Amersham Pharmacia Biotech). YAC end fragments were generated with Alu/Vector-polymerase chain reaction and subcloned in pCR2.1 with the TOPO-TA cloning Kit (Invitrogen). Sequencing was performed as described.

Fluorescence In Situ Hybridization

Metaphase spreads were obtained either from primary blood samples or immortalized cell lines. Preparations were made according to standard protocols (Lichter and Cremer 1992). Cosmid and plasmid DNA was labeled by nick translation with biotin-16-dUTP (La Roche). Slides carrying metaphase spreads were kept in 70% ethanol at 4° C. for one week. 200-300 ng of labeled plasmid or cosmid DNA, 20-30 μg of human Cot-1 DNA (GIBCO BRL), and hybridization buffer (50% formamide, 10% dextran sulfate, and 2×SSC, pH 7.0) were mixed, denatured for 5 min at 75° C. and pre-annealed for 30 min at 37° C. The slides were denatured for 2 min in 70% formamide and 2×SSC, pH7.0, at 72° C. (Ried et al. 1992). The pre-annealed probe was hybridized overnight in a humidifying chamber at 37° C. Slides were washed and stained with avidin-conjugated fluorescein isothiocyanate (FITC). The signal was amplified with biotinylated anti-avidin followed by shining with avidin-FITC. For the probe all human telomeres (Oncor) the instructions supplied by the manufacturer were followed. Chromosomes were counterstained with 4′,6-diamidino-2-phenylindol dihydrochloride (DAPI). Images were taken separately by using a cooled charge coupled device camera system (Photometrics, Tucson Ariz., USA). A Macintosh Quadra 900 was used for camera control and digital image acquisition in the ‘TIF’ format using the software package Nu200 2.0 (Photometrics). Separate gray scale fluorescence images were recorded for each fluorochrome. Images were overlaid electronically and further processed using the Adobe Photoshop software.

Searching the Stature Gene

Microdeletion Screening

Exon Amplification

Shotgun subcloning of PAC clones into pSPL3B. Genomic DNA from chromosome Y specific PAC clones was partially digested with Sau3AI. 100 ng of isolated fragments in the range of 4-10 Kb were ligated with 100 ng of pSPL3B that had been BamHI digested and dephosphorylated. The ligation reaction was transformed into supercompetent E. coli Xl-1 blue cells (Stratagene) and aliquots of each transformation plated on selective medium (ampicillin). Resulting colonies were subsequently pooled for plasmid DNA isolation.

Cell culture and electroporation. COS7 cells were propagated in DME medium supplemented with 10% heat inactivated calf serum. For transfections COS7 cells in between the 5^{th} and 15^{th} passage were grown to about 75% confluence, trypsinized, collected by centrifugation and washed in ice-cold Dulbecco's PBS. 4×10⁹ cells were then resuspended in cold 0.7 ml Dulbecco's PBS and combined in a precooled electroporation cuvette (0.4 cm chamber, BioRad) with 0.1 ml Dulbecco's PBS containing 15 μg DNA. After 10 min on ice, cells were gently resuspended, electroporated (1.2 kV, 25 μf) in a BioRad Gene Pulser 2 and placed on ice again. After 10 min cells were transferred to a tissue culture dish (100 mm) containing 10 ml prewarmed, CO₂ preequilibrated culture medium.

RNA isolation, RT-PCR and cloning. Cytoplasmic RNA was isolated 72 hrs post transfection (QIAGEN RNeasy Kit) and first strand synthesis was performed as recommended by the manufacturer with minor modifications: 5 μg of RNA was added to a solution containing 10 mM of each dNTP and 2 μM of oligonucleotide SA2. The mixture was heated to 65° C. for 5 min and then placed on ice for at least a further minute. After adding a reaction mixture containing 10× PCR buffer (Perkin-Elmer Cetus), 25 mM MgCl₂, 0.1M DTT and RNAsin (35 U/μl), the reverse transcription reaction was transferred to 42° C. for 2 min. 1 μl of SuperScript II RT (200 U/μl; Gibco BRL) was then added and the reaction incubated at 42° C. for 90 min and 50° C. for 30 min. The entire cDNA synthesis reaction was then converted to double strand DNA using a limited number of PCR amplification cycles in the following 100 μl reaction mixture: 1× PCR buffer (Perkin-Elmer Cetus), 1.5 mM MgCl₂, 200 μM dNTPs, 1 μM SA2, 1 μM SD6 and 2.5 U Taq polymerase (Perkin-Elmer Cetus). 6 amplification cycles were used and consisted of 1 min at 94° C., 1 min at 60° C. and 5 min at 72° C. To eliminate vector-only and false positive products, 50 U of BstXI (New England Biolabs) was added directly to the reactions, followed by overnight incubation at 55° C.

10 μl of the digest was then used in a second PCR amplification using internal primers in the following 100 μl reaction mixture: 1×PCR buffer (Perkin-Elmer Cetus), 1.5 mM MgCl₂, 200 μM dNTPs, 1 μM (CAU)₄-SD2, 1 μM (CUA)₄-SA4 and 2.5 U Taq polymerase (Perkin-Elmer Cetus). 25 amplification cycles were used and consisted of 1 min at 94° C., 1 min at 60° C. and 3 min at 72° C. Products were separated by electrophoresis and fragments larger than the pure SD2/SA4 RT-PCR product excised and subcloned (CloneAmp pAMP1 System; Gibco BRL) into pAMP1 according to the manufacturer's protocol. Ligation reactions were then transformed in ultracompetent E. coli XL-2 blue (Stratagene) and plated on selective medium containing X-Gal/IPTG.

Identification of candidate exons. All white colonies were picked and transferred to 384-well microtiter plates containing selective medium and incubated overnight at 37° C. With a 384-pin transfer device 24.5×24.5 cm culture plates with and without positively charged nylon membranes (Amersham) on top of them were inoculated and also incubated overnight at 37° C. Colonies grown on culture plates were pooled for plasmid preparation, colonies on nylon membranes were used for colony lifts. Plasmid inserts were excised, purified, and hybridized to nylon membranes containing EcoRI-digests of the PAC clones used as the original substrate. Highlighting bands were subsequently isolated and hybridized to colony lifts to identify candidate exons. Candidate exons were isolated and sequenced by Sequitherm EXCEL II DNA Sequencing Kit (Epicentre Technologies). Sequences were automatically analyzed and read on an ALFExpress DNA sequencer. Table 6 lists the sequences of the isolated exon trap clones.

Exon Trapping. DNA from chromosome Y specific PAC (P1-derived artificial chromosome) clones RP1-148J07, RP5-1160A12, RP1-301P22, RP4-532107 and RP1-114A11 was partially digested with Sau3AI and fragments in the range of 4-10 Kb were individually subcloned into pSPL3B. COS7 cells were transfected and after 72 hrs cytoplasmic RNA was harvested using QIAGEN RNeasy Kit cDNA synthesis was performed as recommended by the manufacturer (Gibco-BRL). Primers flanking the cloning sites were used to identify products larger than the pure SD2/SA4 RT-PCR product. These fragments were excised, subcloned (CloneAmp pAMP1 System; Gibco BRL) into pAMP1 and sequenced Exon trap clones were labelled with ³²P-dCTP by random priming and used as hybridization probes on Southern blots. Hybridization: 16 hrs at 65° C. in standard hybridization buffer (Singh and Jones 1984). Wash: three times for 20 min each at 65° C. in 0.1×SSC, 0.1% SDS.

In silico gene prediction. Completed genomic sequences from BAC clones RP11-75F05, RP11-461H06, RP11-333E09, RP11-558M10, CITB-298B15 and CITB-144J01 were analyzed for homologies to known genes and virtual gene content using the NIX (http://menu.hgmp.mrc.ac.uk) and Rummage (http://gen100.imb-jena.de) software packages. Computational identification of promoters and first exons was achieved by submitting BAC sequences to FirstEF (http://www.cshl.org/mzhanglab).

Reverse-transcribed polyA⁺-RNAs and cDNA libraries. Human polyA⁺-RNA of 16 fetal and adult tissues was purchased either from Clontech or Invitrogen. Human polyA⁺-RNAs from 3 osteosarcoma and 1 bone marrow fibroblast cell line were isolated by the QIAGEN Oligotex kit. First-strand cDNA synthesis was essentially carried out as described (Rao et al. 1997). Fourteen cDNA libraries were obtained either from Clontech or Stratagene. A collection of 40 cDNA libraries was also provided by the Resource Center of the German Human Genome Project (RZPD). The complete list is available on request.

Characterization of potential transcription units. After homology comparison and open reading frame (ORF) analysis of exon trap clones, primers were designed for RT-PCR amplification. Sequences are summarized in Table 8. In those cases where exon trap clones consisted of only one exon, two exon-specific primers were combined with cDNA-library specific primers in semi-nested PCP, Primers were designed from predicted gene models to amplify across exon/intron boundaries. To provide evidence of transcription, primers were used to screen a panel of cDNA libraries and polyA⁺-RNAs (see above). In the case of potential coamplification from homologous transcripts, primers flanked Y-specific restriction sites.

Evolutionary strata classification. Sequence divergence between genes/pseudogenes of the GCY region and their functional/non-functional progenitors was determined according to Li, 1993. Sequences for all pseudogenes were extracted from genomic sequences: KIAA1470PY from BAC clone RP11-75F05 (AC011293), KIAA1470P1 from BAC clone RP11-498M14 (AL445675), ADLY from BAC clone RP11-333E09 (AC011302), ARSFP and RPS24P1 from BAC clone CITB-144J01 (AC004772), RPS24PX from BAC clone RP11-418N20 (AC119620), ASSP6 from BAC clone RP11-461H06 (AC012502) and ASSP4 from BAC clone GS1-536K07 (AC004616). Sequences for all other genes were obtained from published cDNAs, whose GenBank accesion numbers are as follows: ADLX (AF245505), ARSF (XM_{—}035467), RPS24 (NM_{—}033022), ASS (X01630), KIAA1470 (AB040903). THC604695PY was not analyzed as only part of its most terminal exon (consisting almost entirely of 3'UTR) was available for comparison with the X-chromosomal EST cluster (AA662182 and AA662138).

### RESULTS

Mapping of interstitial deletions

We studied the DNA of nine adult males which originally consulted reproduction centers about idiopathic infertility, but were otherwise generally healthy. Of the 9 males, 7 were unremarkable with respect to adult height. One patient, #293, with a height of 157 cm, presented short stature (SDS −2.9) and one, Y0308, with a height of 165.5 cm showed borderline height, being at the 3^{rd} percentile of normal U.S. height standard (SDS −1.7). Adult height of his parents and siblings are in the normal range (Table 1), his brother being 20.5 cm taller than the patient Compared to his target height (178 cm) and target range (169-187 cm) he can be considered short. All men were ascertained solely on the basis of the occurrence of large de novo interstitial deletions on the Y chromosome. Only two of those patients had undergone previous chromosomal studies.

In our effort to localize the GCY locus, we focused on that part of the Y chromosome long arm, which was delimited by the boundaries of the interstitial deletions of the patients with short stature (FIG. 1). Recently, a detailed physical map of the human Y chromosome incorporating 758 ordered STSs and 199 completely sequenced BAC clones has been constructed (Tilford et al. 2001). We used a slightly modified PCR multiplex system (Henegariu et al. 1994) to test the absence or presence of 28 DNA loci from the Y chromosome long arm. In patients where sufficient DNA was available for further PCR analysis additional STSs were tested. As a result, 8 of 9 interstitial deletion breakpoints could be positioned (FIG. 1). As the deletions of patients JOLAR, #28, #63, #95, T.M., and #1947, all with normal height, overlap, most of the long arm of the Y chromosome could be excluded as a critical region for GCY.

As the distal breakpoint of the deletion of patient #1972 does not reside within the specific part of the Y chromosome long arm, the nature of the deletion (terminal or interstitial) remained unclear. There was also no overlap of his deletion with the deletions of patients #1947 and T.M. Relying solely on the results obtained by the STS-based interstitial deletion mapping strategy, one could not formally exclude the region distal to sY158 as a potential critical region for GCY. However, multiplex PCR analysis always showed a less intense amplification product for STS sY157 (a Y-derived marker in close vicinity of sY158). To address this problem, the rearranged Y chromosome of patient #1972 was investigated in more detail.

Fluorescence In Situ Hybridization and Sequence Family Variant Typing Of Patient #1972

The overall integrity of the Y chromosome from patient #1972 was demonstrated by FISH of the cosmids LLOYNC03“M”34F05 (PAR1) and LLOYNC03“M”49B02 (PAR2) as well as the Y-centromere-specific probe Y-97 and the telomere-specific probe ‘all human telomeres’ (data not shown). Being aware of the complex structural organization of the human DAZ locus (FIG. 2A), we specifically searched for sequence family variants (SFVs). To prevent misjudging sequence errors as single nucleotide differences, PCR/restriction-digestion assays were developed only from SFVs present in at least two overlapping BAC clones. The localization of these SFVs is shown in FIG. 2B. As these SFVs could represent allelic variants, ten unrelated normal German males were typed. In all cases, the expected fragment pattern could be detected for the Y-chromosome derived sequences. In contrast, the fragment pattern deduced from the genomic sequence of the chromosome 1-derived BAC clone RP11-560118 could not be confirmed (see Table 3 for detail). Each SFV-specific PCR/restriction digestion was compared to the presence/absence in the corresponding BAC clones.

Typing the genomic DNA of patient #1972 for all four sequence family variants (SKY10/Tsp509I, SKY11/NlaIII, SKY12/MseI, and SKY13/Cac8I+TfiI) revealed the absence of one Y-derived non-allelic sequence variant (Table 3 and FIG. 2C,D). In the case of SKY10 the distal copy is deleted. Not surprisingly, in all other typing experiments the more proximal copy of the respective SFVs was shown to be deleted.

Next, we investigated these SFVs in the two patients with the most distal breakpoints (#95 and #1947). Using genomic DNAs, we determined that both non-allelic variants of SKY11, SKY12, and SKY13 and one non-allelic variant of SKY10 were absent in patient #1947, whereas for all tested SFVs one non-allelic variant was absent in patient #95.

Taken together, these results provide evidence that the proximal breakpoint of the interstitial deletion present in the Y chromosome of patient #1972 resides within the interstitial deletion of patient #1947, thereby excluding this genomic region as a potential critical interval for GCY.

Refinement of the GCY Critical Interval

Based on the molecular analysis of the pericentric region of the long arm of the human Y chromosome (Williams and Tyler-Smith 1997), the physical extension of the GCY critical region as defined by the markers sY78 (DYZ3) and sY83 (DYS11) was estimated to constitute 1.6-1.7 Mb (FIG. 3A) of DNA. The most proximal 400 kb of this region consist exclusively of 5 bp satellite sequences separated from the Y centromere only by Alu sequences. This constant part of the human Y chromosome is therefore unlikely to contain coding sequences. The remainder of the GCY critical region is composed of X/Y-homologous as well as autosomal/Y-homologous sequence blocks. At the onset of this study, only limited coverage in YAC clones was available for this region. In order to refine the GCY critical interval and to generate gene finding substrates, it was necessary to establish a BAC/PAC-contig of this region.

We generated 25 additional markers mainly by sequencing the end fragments of BAC, PAC, and YAC clones as well as clone-internal sequences amplified by various combinations of Alu-Alu oligonucleotide primer pairs. Of those, only 7 turned out to be Y-specific (SKY1, SKY2, and SKY4-8) (see Table 2 for detail). The BAC and PAC clones identified during the generation of the physical map are summarized in Table 4. Meanwhile, some of these clones have been completely sequenced as they form part of a tiling path for sequencing the human Y chromosome (Tilford et al. 2001). The proximal part of the cloned region between markers sY78 and SKY6 has not been sequenced to date. A selection of clones covering the entire GCY critical region is depicted in FIG. 3.

Confirming the overlap between BAC RP11-295P22 and BAC RP11-322K23 appeared to be the most crucial step in the process of contig construction. Y-specific markers derived from the opposite end fragments of both clones were suspected to amplify identical-sized fragments from two different loci within the same 5 bp satellite region. By testing several restriction enzymes known to cut frequently within 5 bp satellites composed of the consensus sequence (TGGAA)ₙ, we developed loci-specific PCR/restriction digestion assays. Typing all BAC clones mapping to this sequence block with the appropriate PCR/restriction digestion assay allowed us to precisely position them thereby confirming their overlaps.

In order to narrow down the critical interval for the GCY gene, we tested for the presence of the newly generated STS in patients #293, Y0308, and JOLAR. These results allowed us to define a small region for the GCY gene (FIG. 3 and FIG. 4). Direct sequence comparison showed that the sequenced BAC clones RP11-322K23, RP11-75F05, RP11-461H06, RP11-333E09, RP11-558M10, CITB-298B15, and CITB-203M13 completely cover the mapped region between Y-STSs SKY8 and sY83 (DYS11), suggesting that it encompasses roughly 700 kb. Basically, the region can be subdivided in three distinct intervals: a proximal region characterized by 5 bp repeats, a central region with high homology to chromosome 1, and a distal region composed of X(Y-homologous sequences. As the most distal part of the GCY critical region (beginning with bp1 of BAC clone CITB-144J01) was already subject of extensive research during the process of characterization of the AZFa critical region and was shown to harbour no functional gene (Sargent, et al. 1999), it was excluded from further detailed genomic DNA analysis. The most proximal part of the GCY critical region consists exclusively of satellite type 3 sequences of the 5 bp consensus (TGGAA)ₙ and is therefore also not assumed to contain any gene. Leaving these two regions out of consideration, we were able to concentrate our efforts to a smaller interval of 420 kb of DNA. Large-scale sequence comparisons performed by the Advanced PipMaker software showed no integration of Y-specific sequences into the chromosome 1 and/or chromosome X-homologous regions.

We have also established new Y-specific markers scattered uniformly across the entire 420 Kb of DNA (Tab 5).

Exon Trapping in the GCY Critical Region.

The boundaries of GCY region are defined by two deletion patients, JOLAR and Y0308 (FIG. 3). PAC clone, RP1-148J07, extends into a genomic segment exclusively composed of 5 bp repeats of the satellite 3 type. The very distal PAC clone, RP1-83D22, was not included in the experimental analysis, as the region distal to sY82 was previously analyzed in the course of defining the transcriptional potential of the AZFa region (Sargent et al. 1999). To identify transcripts that might encode GCY, we used 5 PAC clones from the GCY region as substrates for exon trapping (RP1-148J07 up to RP1-114A11, FIG. 4). Each of the 5 PAC clones from the GCY region was individually subcloned and subjected to exon trapping. Nucleotide sequencing of trapped products identified 9 different exon trap clones, two of them were composed of two exons (FIG. 4, Tab. 6). All exon trap clones were isolated in several copies. Exon/intron boundaries of all 11 putative exons matches the splice site consensus. Trapped products that mapped to the GCY region were verified using PCR by their presence versus absence in males and females and GCY-deleted males with short stature. All exon trap clones revealed only one male-specific fragment on Southern blots.

In Silico Analysis of Annotated BAC Clones.

We analysed the genomic sequence of the complete GCY region using the gene prediction programs assembled by the NIX and Rummage software packages. Homologous sequences were also analysed in the non-redundant (nr) database of GenBank using the BLASTN or FASTA algorithm. BAC RP11-75F05, for example, includes a 1 Kb segment with a 77% homology to the transcriptional unit KIAA1470 on chromosome 1p36 (FIG. 5). On BAC RP11-461H06 and CITB-144J01, for example, sequences of 2.5 and 1 Kb length showed a 88% and 81% homology with the genes ASS and RPS24 on chromosome 9q34 and 10q22, respectively. The Y-chromosomal copies ASSP6 and RPS24 P1, however, represent pseudogenes and have a progenitor on Xp22 that has been translocated to the Y chromosome. Two pseudogenes on RP11-333E09 and CITB-144J01, THC604695PY and ARSFP, represent deleted copies of Xp22 specific genes.

BAC RP11-333E09 includes a deleted duplication (ADLY) of the adlican gene on chromosome Xp22 (ADLX). ADLX has been previously shown to be upregulated in osteoarthritic tissue and therefore likely plays a role in bone metabolism. The Y chromosome copy, therefore, constitutes an important candidate for a gene involved in growth. Despite the loss of exons 3 and 4 as a consequence of intrachromosomal recombination, its basic structural organization (FIG. 7) and sequence homology to ADLX (Tab. 9a) could still allow to encode a functional protein with similar molecular properties. This observation was enforced by a unified predicted gene model of ADLY by all gene-finding programs (cf1; FIG. 4). Taking the functionality of the predicted ADLY promoter for granted and assuming ADLY would start at the ATG codon also used on the X chromosome, an in-frame stop codon at position +359 would result in premature termination. One additional promotor was predicted in the sense strand of the last intron of ADLY. There is, however, no obvious correlation between the promoter position and the significance for potential ADLY expression.

Using various gene-finding programs we detected 17 gene models in the GCY region (FIG. 4f). Only five (ar1, cf1, cr1) overlapped with transcriptional units identified by homology search. Conceptual translations of 14 models revealed no protein matches. With respect to location and orientation promoters predicted by FirstEF could be assigned to KIAA1470P, ADLY, RPS24P1, and ARSFP.

In conclusion, there is no identity of exon trap clones and gene models/homologies or pseudogenes KIAA1470PY, ASSP6, and THC604695PY. Considering ADLY as the most attractive candidate for the GCY locus, we directly compared the exon/intron boundaries of the Y- and X-derived copy (Tab. 9b). Exons 3 and 4 of ADLX are deleted on the Y copy. The remaining 3 internal exons still possess correct 5′ and 3′ splice sites.

Searching for a Transcriptional Unit

Homology searches performed with all exon trap clones and predicted gene models against the dbEST segment of GenBank did not yield any Y-specific EST. PCR and PCR/restriction digestion assays with primers corresponding to all putative transcriptional units were carried out. Primers derived from all exons of ADLY (Tab. 7B, 7C), the most prominent GCY candidate, were used to screen reverse-transcribed polyA⁺-RNAs from osteosarcoma and bone marrow fibroblast cell lines. Whereas ADLX was shown to be expressed in all tested cell lines (with the exception of neuronal tissues), no ADLY specific specific transcript was detectable. More extensive screening of polyA⁺-RNAs from various adult and fetal tissues basically led to the same result. We also tested all putative transcriptional units in the GCY region for expression in polyA⁺-RNAs from 21 tissues and 49 cDNA libraries. RT-PCR assays did not provide proof of a transcribed gene.

Evolutionary Features of Time GCY Critical Region.

High sequence homology of the Y chromosome to other chromosomal regions is consistent with an evolutionarily recent transposition of those regions to the Y chromosome. More subtle nuances in synonymous nucleotide divergences of homologous gene pairs (K) allow their integration into distinct evolutionary strata, group 1-4 (Lahn and Page 1999). The calculated Kₛ values for all gene pairs in the GCY region along with Kₛ values from reference genes of the different stratas are given in table 6. We noted that the Kₛ values for all X-Y gene pairs can be grouped into the most recent evolutionary stratum (group 4), having been embarked on X-Y differentiation 30 to 50 million years ago. This classification is independent of the actual functional state of X-chromosomal genes. Comparing Kₛ values between the Y-copies in the GCY region and their functional progenitors clearly demonstrates that decay of the X-chromosomal copies took place before the X-Y recombination occurred. Even more prominent is the difference between Kₛ values for the chromosome 1-chromosome Y gene pairs. The low Kₛ value for the KIAA1470P1/KIAA1470PY gene pair points towards a very recent transposition to the human Y (FIG. 5). Supporting evidence comes from fluorescent in situ hybridization in primates delineating this event to a time period of about 5 to 6 million years ago (Wimmer et al. 2002). The Kₛ value for the comparison of KIAA1470PY with its functional progenitor in 1p36 date the underlying intrachromosomal transposition roughly to about 150-170 million years ago.

As the frequency of nonsynonymous substitutions (Kₐ) is a function of both evolutionary time and selective constraints on the encoded proteins, the degree of constraint can be reflected in the ratio Kₛ/Kₐ (Li, 1993): Values greater than one indicate the presence of constraints on both homologs, and values in the vicinity of one are consistent with lack of constraint on at least one homolog. All determined Kₛ/Kₐ ratios suggest that natural selection on the Y copies is not ongoing thereby underlining their pseudogene status.

We searched the nr database of Genbank with the homology transitions and the distal border of the GCY region to precisely determine the physical extent of the homologous regions on chromosomal subintervals 1q43 and Xp22. To identify highly conserved segments, we used Advanced PipMaker (Schwartz et al. 2000, http://bio.ces.psu.edu) for comparing the corresponding DNA. Inspection of the compound dot plot allows the identification of those portions of the GCY region absent in homologous sequences. As the overall homology of Y/1 and Y/X in conserved regions is already in the range of 94-97% and 96-99%, putative protein-coding exons are not expected to show average percent identities higher than the non-coding environment Careful dot plot analysis showed that all novel sequences that have accumulated in the GCY region on the Y after the separation from its autosomal or X-chromosomal counterpart are exclusively of repetitive origin. Particularly evident is the prevailing preponderance of integrated LINEs family members.

Discussion

Since the issue on the existence of a Y-specific growth gene (GCY) was first raised, there have been several attempts to define its precise location. Whereas initial studies unanimously pointed towards a common region of the Y chromosome long arm (Salo et al. 1995), more recent investigations have led to the identification of two non-overlapping critical intervals (Rousseaux-Prevost et al. 1996, Ogata et al. 1995, De Rosa et al. 1997). FISH analyses resolved this apparent contradiction by presenting clear evidence that the patient materials used in these initial investigations contained 45,X0 cells and/or i(Yp) or idic (Yq11) chromosomes (Kirsch et al. 2000). Both genetic parameters influence the adult height of a given individual, thereby rendering it impossible to predict whether such patients have lost GCY or not Studies with patients carrying de novo interstitial deletions are, therefore, much better suited to address the problem of GCY localization.

In the course of winnowing the literature for patients with small interstitial deletions, in particular close to the centromere, it became clear that those patients are very rare. This prompted us to extend our search for patients carrying large de novo interstitial deletions, irrespective of their actual adult height. We examined 9 adult patients, 7 of whom presented normal height Furthermore, we could show overlapping deletions, thereby excluding GCY to reside between the Y-specific marker DYS11 and the pseudoautosomal region 2 (PAR2). Two patients, #293 and Y0308, presented interstitial deletions enabling the restriction of the GCY critical region to approximately 700 kb of DNA. This region is therefore predicted to harbour one or more genes required for normal human growth.

Exon Amplification and Gene Modeling in the GCY Region.

Although much attention has been drawn to the various azoospermia (AZF) critical regions in Yq11 as well as Y-encoded testis-specific or ubiquitously expressed genes, the GCY region up to now was not searched systematically for transcription units. We have used exon amplification, homology search, and in silico gene prediction to identify putative genes within this region. This information now provides the means to test candidate genes for involvement in human linear growth regulation. Up to date, the major problem in defining the GCY gene was the lack of potential transcription units assigned to this portion of the human Y chromosome. Prior to this study, there were only two pseudogenes, RPS24P1 and ARSFP, that mapped to the GCY critical region (Sargent et al. 1999).

By exon amplification we isolated 9 different exon trap clones, two of which were composed of two exons. Parallel sequencing efforts of the GCY region by the Human Genome Project allowed us to complete our catalog of potential transcription units in the GCY region. No Y-specific ESTs were assigned to the region. The Nix and Rummage software programs were used to analyze sequence data of completed BACs to predict potential genes in the sequence. We have identified 4 new genes/pseudogenes and 17 gene models. Of the 17 gene models, only five have homologies to the identified genes/pseudogenes. A gene model homologous to ADLY (cf1) was uniformly predicted by all gene-finding programs. Though, the probability given by various gene finding programs might be overestimated with regard to the gene model cf1. Very large exons, as present in ADLY, are less likely to be predicted correctly, but they are most unlikely to be completely missed. Consequently the tendency to classify actual pseudogenes as functional genes increases with the presence of large exons. The failure to trap exons of the putative ADLY transcription unit, albeit possessing correct splice sites, might be an intrinsic feature of Y-chromosomal sequences. Complete representation of the AZFc region in cosmid/P1 clones used for exon-trapping experiments (Reijo et al. 1995) led to the detection of DAZ as the only gene out of a possible 8 genes/gene families located in this region (Kuroda-Kawaguchi et al. 2001).

Surprisingly, we observed no concordance between the gene models and the exon trap clones. It is possible that exon amplification is dependent on the presence of functional splice sites in the genomic sequence whereas gene modeling is mainly based upon the in-phase hexamer measure (Rogic et al. 2001), a method determining the incidence of oligonucleotides of length six in a specific open reading frame. On the other hand, the prediction of correct splice sites is less important since such signal sensors have low information content and are usually degenerate. Consequently, the exon trap clones need not to be necessarily part of one of the predicted gene models, although a substantial fraction of the trapped exons (7/11) are composed of 75 to 200 nucleotides, a length range in which exons are most accurately predicted. Likewise, the putative exons assembled to a distinct gene model do not necessarily represent real exons.

It is possible that the eventual number of genes in the GCY region is smaller since exon trap clones and/or gene models turn out to be part of the same transcripts or do not represent genes at all. Despite the number of potential transcription units in the region, however, the search for the critical one might still be complicated by the fact that the phenotypic effect caused by mutation of the GCY locus is hard to be defined precisely. This makes it difficult to predict an expression profile, especially when the gene function is unknown. Since human linear growth is a multifactorial trait, growth failure is quite common. Although at least nine growth-controlling genes have been identified up to now, only few cases present disease-causing mutations within those genes. Definition of the transcription units in the region should now facilitate mutation studies, especially since full-length genes/pseudogenes have been isolated

Although reverse-transcribed polyA⁺-RNAs and cDNA libraries have been extensively screened, we have not detected any transcript specific to the Y. This raises the question whether our approach was suitable. To assess its usefulness we have verified the expression pattern of 20 genes known to be essential for bone development at GenePage (http://genome-www5.stanford.edu). At least double presence for each selected gene was warranted by our screening efforts. This corroborates the existence of an unusual gene with an extremely confined spatial and/or temporal expression pattern.

Evolutionary Features as a Clue to the GCY Locus?

To gain more insight into the molecular genesis of the GCY critical region, we used two methods. First, we validated the functional state of the genes/pseudogenes within the GCY region by comparing them with their direct and functional progenitors. All gene pairs showed Kₛ/Kₐ ratios of 1 to 2 rather indicating that the Y copy is a pseudogene. This result assigns the X-Y gene pairs to evolutionary stratum 4 which fits very well since all those gene pairs share a common evolutionary history. Only one gene pair out of this class, AMELX/Y, still encodes a functional X- and Y-copy (Salido et al. 1992). The Y-copy of KIAA1470 clearly could be classified as a pseudogene by comparing it with its functional progenitor on 1p36. Second, we made use of large-scale sequence comparison in order to identify potential differences between the subintervals of the GCY region and their homologous counterparts in Xp22 and 1q43. Neither subregions with a conservation level above the molecular environment nor small genomic fragments newly integrated into the GCY critical region could be detected. Furthermore, promoter prediction carried out simultaneously on homologous genomic sequences revealed no differences. This clearly excludes substantial rearrangements within the GCY critical region and lends support to a gene underlying male-specific regulatory mechanisms.

TABLE 1
Adult height comparison of patients and their siblings

| | | Height of patient | National | Heights of family |
|---|---|---|---|---|
| | | (cm) and | height | members (cm) and |
| | Country | standard deviation | standard | standard deviation |
| Case | of origin | score | (cm) | score |
| #293 | U.S.A. | 157 (SDS −2.9) | 176.9 | (F) 170 |
| | | short | (SD 6.8) | (M) normal |
| | | | | (B) normal |
| Y0308 | U.S.A. | 165.5 (SDS −1.7) | 176.9 | (F) 170 |
| | | borderline | (SD 6.8) | (M) 168 |
| | | (short?) | | (B) 188 (SDS +1.7) |
| | | | | (S) 170 (SDS −0.4) |
| JOLAR | United | 168 (SDS −1.0) | 174.7 | (F) normal |
| | Kingdom | normal | (SD 6.7) | (M) normal |
| | | | | (B) normal |
| #28 | Italy | 175 (SDS −0.3) | 176.7 | (F) normal |
| | | normal | (SD 6.5) | (M) normal |
| #63 | Ethiopia | 170 (SDS +0.3) | 168.0 | (F) normal |
| | | normal | (SD 7.4) | (M) normal |
| #95 | Israel | 185 (SDS +1.4) | 175.6 | (F) normal |
| | | normal | (SD 6.8) | (M) normal |
| T.M. | Belgium | 182 (SDS +1.3) | 173.5 | (F) normal |
| | | normal | (SD 6.7) | (M) normal |
| #1947 | Germany | 175 (SDS −0.8) | 179.9 | (F) normal |
| | | normal | (SD 6.4) | (M) normal |
| #1972 | Germany | 181 (SDS +0.2) | 179.9 | 175 (F) |
| | | normal | (SD 6.4) | 165 (M) |
| | | | | 172 (S) (SDS +1.0) |

The standard deviation score (SDS) was calculated based on the equation: SDS = (X − M)/SD, where X is an individual's adult height and M and SD are the mean adult height and the ±1 standard deviation of the normal population, respectively.
(M) mother,
(F) father,
(S) sister,
(B) brother,
(NA) not available.

TABLE 2
Y-chromosomal STSs

| | | | |
|---|---|---|---|
| STS | Left Primer | Right Primer | Product |

| | | | |
|---|---|---|---|
| SKY1 | GGACATTTGGCTGCAGAGAT | TGGCAATGCACTCTCATCAT | 255 |
| SKY2 | TCAGGACAGACAGGCTGCTA | CCTGCCACTGAGCTCCTTAC | ˜1700 |
| SKY3 | TTCTCCCTCATCTTCCAAGC | GCTTCCATCCATTAGCAAGG | 167 |
| SKY4 | CCTTTCATTCCATTCTCTTCCA | CGCACTTTATGGACTGCAA | 111 |
| SKY5 | CCCTCGTCCATTTCTTTTGA | CCTCGAATTTAATGGATTGC | 202 |
| SKY6* | TCAATGGATGCACAGTGTGGC | TCCACTGAATTCCATTGCAC | 328 |
| SKY7 | GGGAGTGCAAAGGGAAAGAT | CTTTCCATGGGGTGACATTC | 223 |
| SKY8 | CCATTCATTCGAGTTCATTACG | ATTGGAATGGAATCGGACAG | 189 |
| SKY9 | GGCCGATGGTCAAACTGTTA | GAAACGGGCTCGAAATTCT | 531 |
| SKY10* | ATAAGGGGCAGGTTTGTCAC | GCTACTTATTCAGTGTTTAACTGACAC | 329 |
| SKY11* | AAAGTGGGTGAAGGACATGG | TTTTTGTTTGTGGCAGGTG | 469 |
| SKY12* | TTGAGTCACTGGGGATAACTG | TATGGCCCACAATCACTTCA | 216 |
| SKY13 | GGCAGCCTAGAAAGTCTTGTTC | CCCTTGGGATTTTGTCTGTT | 198 |

Markers indicated with a * amplify DNA fragments from more than one genomic locus (see Chapter Restriction analysis of PCR products for detail).

TABLE 3
PCR/Restriction Digest Analysis of Sequence Family Variants in the AZFc

| | Restriction | BAC | Fragment sizes (bp) |
|---|---|---|---|
| STS | enzyme | clones | after restriction |
| SKY10 | Tsp509I | 487K20 | 279, 50 |
| | | 70G12 | 329 |
| | | 560I18 | 329* |
| SKY11 | NlaIII | 245K04 | 217, 154, 79, 19 |
| | | 506M09 | 233, 221, 15 |
| SKY12 | MseI | 245K04 | 88, 57, 39, 32 |
| | | 506M09 | 145, 39, 32 |
| SKY13 | Cac8I/TfiI | 100J21 | 97, 83, 23 |
| | | 589P14 | 175, 23 |
| | | 251M08 | 97, 50, 33, 23 |
| *The submitted sequence of the chromosome 1-derived BAC clone RP11-560I18 (AC053522) does not show a Tsp509I restriction site within the genomic fragment amplified by the primer pair SKY10. Restriction analysis of fragments amplified from male and female genomic DNA, from a somatic cell hybrid line containing chromosome 1 as the only chromosome of human origin and from the BAC RP11-560I18 as well | | | |
| # shows two fragments of ˜180 bp and ˜155 bp indicating a sequence error in the complete sequence of the BAC clone. | | | |

TABLE 4
Summary of BAC and PAC clones identified during physical map creation

| Y-STSs | Positive BACs (RPCI11) | Positive PACs (RPCI1, 3-5) |
|---|---|---|
| sY83 | not screened | 83D22 |
| sY82 | not screened | 83D22, 114A11, 157G08, 966C15 |
| GY8 | not screened | 114A11, 168E21, 271D03, 635F21, |
| | | 765H16, 806O15, 904E13, 966C15 |
| sY81 | not screened | 301P22, 1079J08, 1078C20, 1160A12 |
| 14A3C* | not screened | 148J07, 1136A14, 1160A12, 1196I23 |
| sY79 | 75F05, 79E14, 102G24, 322K23, | 1149H11 |
| | 417D23, 600D11, 612E10, 725I12, | |
| | 863I08, 903M02, 1125H21 | |
| SKY1 | 376B16, 544C11, 544M21 | 56A05, 85D24, 958M03 |
| SKY2 | 79P12, 295P22, 376L20, 828O24, | 829H08 |
| | 886I11, 910C06 | |
| SKY4 | 75F05, 322K23, 612E10 | not screened |
| SKY5 | 174I24, 271E18, 295P22, 588E18, | not screened |
| | 620J20, 632F11, 684H19, 705O19 | |
| SKY6 | 174I24, 271E18, 295P22, 588E18, | not screened |
| | 620J20, 632F11, 684H19, 705O19 | |

*14A3C is a hybridization probe previously described by Tyler-Smith et al. 1993. It detects a Y-specific HindIII-fragment of 3.5 kb and an additional autosomal fragment.

TABLE 5

Genomic primer pairs for microdeletion screening
in adult males with idiopathic short stature

| Primer sequence (5′→3′) | | product | | genomic location* | |
|---|---|---|---|---|---|
| forward | reverse | size | primer | forward | reverse |

| | | | | | |
|---|---|---|---|---|---|
| ATTTCCACCGAAACCCATTT | CTCCCCTACCACCAACACAC | 251 | A72 | 72300-72318 | 72549-72530 |
| AGGGCCCTCACATGATTAAA | GCGACACCATTTCTTTCCAT | 255 | A92 | 91949-91968 | 92204-92185 |
| GACATCGTGGTGTCTGTTGC | CAGACGTTGTTCAGGTCGTG | 232 | A111 | 111509-111528 | 111740-111721 |
| GCACCATTAGTGCGCTTGT | TTCTCCCTTTACCCCAAATTC | 269 | A134 | 134542-134560 | 134810-134790 |
| CCAGCAGGAGTCTTGGAGTC | TGAGAGGCACCTACGGTTAGA | 250 | A158 | 157911-157930 | 158160-158140 |
| CCAAGCATGCCTTCCTAAAG | TGCCTTCTCATCTGCTTGTG | 147 | B17 | 17598-17617 | 17744-17725 |
| ATCCTGGGAGATGCATCAGA | TGAGTCCTAAACCGTACACATACA | 209 | B37 | 37406-37425 | 37614-37591 |
| b_r_002for | | | | | |
| CAATGGAAATGTTGCAGGTG | TCCTGCCCTGCTGTTAGAGT | 158 | B59 | 59871-59890 | 60028-60009 |
| GCAAGGGTGTTGCAAGTTTA | TGCATATTGTCCACACATGG | 360 | B82 | 82128-82147 | 82487-82468 |
| AAAGAGAAGGGCCCTGTGAT | CTAGGCAACAGCACTGGAAA | 239 | B102 | 102854-102873 | 103092-103073 |
| AAAATCCAACTTCCCCAGTG | GCAAGAATCTGGGCTCTCAC | 353 | C17 | 17307-17326 | 17659-17640 |
| | c_f_001rev | | | | |
| CACTGGGGAAGGCTGTGATA | CATTGTCATCACTGCCAGGT | 339 | C37 | 37271-37290 | 37609-37590 |
| CCCACTTCTTCTCCAAAGTCC | GCACCCGTTTTTCCTGATCTA | 139 | C56 | 56159-56179 | 56297-56278 |
| c_r_005rev | | | | | |
| GGGGCATATTCTACACACCAA | TGAAATGGCAAACCTTTCAGA | 495 | C77 | 76731-76751 | 77225-77205 |
| | et_c_003rev | | | | |
| AAGAATGGAAGGATCTCCAAGA | TCTGTGCAGAAATGATGGATTC | 342 | C97 | 96759-96780 | 97100-97079 |
| TGGTAGTGGGAAACTGCTCA | TGGTGTGCTAAGTGGCTGTC | 144 | C120 | 120709-120728 | 120852-120833 |
| c_r_003rev | | | | | |
| GCTGCAGTTAGCTAAACCAAGAC | ATTCTGCCTGAACCTCCAGA | 162 | C142 | 142289-142311 | 142450-14243 |

TABLE 6

Sequences of isolated exon trap clones
Exon trap clones:

| | | | |
|---|---|---|---|
| | | Size | |
| Name | Sequence (5′→3′) | (bp) | Orientation |

| | | | |
|---|---|---|---|
| et_a_001 | GGTCTTTGGCTCAACTCAGGTTCCCTCTACCTGAAATGATCCACCTTCAGAGAATTGGATG | 61 | reverse |
| et_a_002 | CTGTGTTGCCTCCTCGATGGGAAAAGAAACAAGCGCACTAATGGTGCATTT (exon 1) | 182 | reverse |
| | CTGGAGCATCAGGGGTGTCTTCTATGATCAAGGAAGGAAGCCACTCAGGGTGATAGAGCT | | |
| | GCAGACTTCTGCTTGGTCACTCTGATAGCTCTGGGAACACTGTGCACCTCTCTGGCTGTG | | |
| | ATGGGGAAACT (exon 2) | | |
| et_a_003 | CTTTTACATAGAATGGTAACTCCTTTTGCACCTCGTGTTTTTTC | 44 | forward |
| et_a_004 | AAAGTTGGTAGTTCGCTCCCGGGCTGATGCTCAGAGTGTGGAACTTGAGGAGCTGCGGTG | 171 | reverse |
| | ACATCCTGCAGCCACACGGGAGGTGGCTCCTCAGGGGCGATTGCTGGCTGTGTCACCACC | | |
| | AGGGGACACCGGGCACAGCTTGAAGCTTGGGGACAGGGAGCTGAGAGGCAC | | |
| et_c_001 | GATTACATGGACTACTATATTTAAAATTCCTTCTAAACTTTTTCCCATTTCTGCTCAATT | 93 | forward |
| | TTCATTCTCCAATATTTGCAAAACTTAAAGTTC | | |
| et_c_002 | GCTGAACATTATTTCTTTATTCCAGATTAGAGGACTAGGATTCATGGGATTATGCATCAA | 60 | forward |
| et_c_003 | GGAAATCTTGAAATGGCAAACCTTTCAGAAGAGATGGCAGAGACTCTCCTACATATTCTG | 68 | reverse |
| | TTCTCAAT | | |
| et_e_004 | ACACTGGAAGAATTGGTGTCTAGGCAGTCTGGGATAATAGCCTAGTTCTAAGGACATTAT | 188 | reverse |
| | CATTGATCCCTTTATAGGCCATAGACCTCCAT (exon 1) | | |
| | TTCTTCCTGTTGGTGCAGGAGGGTGATTAAGGGCTTTTCCTACCTTAAGTTGATCAAAGT | | |
| | GGTATTTTCATAAGATTAATCTGGCAGCAGAATGCA (exon 2) | | |
| et_c_005 | CTTGGTTGGGAAAATATGGCCACCATATTGCTGGGAAAGCCACCAAGAGTGGACTGTTAC | 79 | forward |
| | CAATATCCAAGGGACATGA | | |

TABLE 7A
Primer pairs for predicted genes

| Primer pairs for predicted genes | | product | predicted | restriction | genomic location³ | |
|---|---|---|---|---|---|---|
| forward | reverse | size¹ | gene | enzyme² | forward | reverse |

| | | | | | | |
|---|---|---|---|---|---|---|
| GCTTGGAACTTGAGGTGCTC | GGAGATGTGGGCTTGTGAGT | 482 | a_r_001 | | 104600-104581 | 103332-103351 |
| CTGTGGGTGCATTAGGTGTG | CTGGTACATGCTGCCTGCT | 841 | a_r_002 | | 144939-144920 | 111361-111379 |
| GACCTCTTTTGAGAAAGTCAGCA | AAAGCAATGGCAACAAAAGC | 446 | b_f_001 | | 30214-30236 | 61274-61255 |
| AGAGGGAGGAAAGAGCCATC | GTTGTACGGGCTGCAGAATC | 790 | b_r_001 | | 25244-25225 | 762-781 |
| TGAGTCCTAAACCGTACACATACA | TTTCTGTGCGTGAGAACACA | 122 | b_r_002 | | 37614-37591 | 29995-30014 |
| TCTCTGTGGTGCTGATCCTG | GCAAGAATCTGGGCTCTCAC | 730 | c_f_001 | | 6243-6262 | 17659-17640 |
| ATCCCTATTCGCCCCTTAGA | | | c_f_001b | | 10734-10753 | |
| ACCTCAGGGTGCAGCTTTTA | TGAGCAGTTTCCCACTACCA | 350 | c_f_002 | Bsh1236I | 80230-80249 | 120728-120709 |
| GCTGCAGTTAGCTAAACCAAGAC | TTCTGCAAGGGTCTGGTTCT | 123 | c_f_003 | A1wI | 142289-142311 | 162171-162152 |
| CACAGAAGCCAGGGATCG | GCATCTCGCCCTTTCCTC | 1150 | c_r_001 | BamHI 6361-6344 | 2888-2905 | |
| CAACACTGTACACCGCAACA | TTCTCCAAAGTCCGATACCTG | 172 | c_r_002 | BspMI | 81022-81003 | 56167-56187 |
| TGGAGACATTCACAACGTCAA | TGGTAGTGGGAAACTGCTCA | 325 | c_r_003 | A1uI | 129988-129968 | 120709-120728 |
| AGCTGCCTGACTTCTTGGAA | CTTGCCCACACCTTGATCTC | 574 | c_r_004 | AccI | 170431-170412 | 162765-162784 |
| CGTGCTGGATTCCTATTTGG | CCCACTTCTTCTCCAAAGTCC | 212 | c_r_005 | MspI | 66318-66299 | 56159-56179 |

¹predicted product size in bp;
²Potential Y-derived transcript copies will be cut with the indicated restriction enzyme, potential X-derived transcripts remain uncut;
³indicates primer positions (orientation centromer to telomer) in the predicted gene containing BAC (a, b, c or d).

TABLE 7B
Primer pairs for Y copy of Adlican

| | Direction with | |
|---|---|---|
| | respect to | |
| | putative tran- | |
| Primer sequence | scription | |
| (5′→3′) | orientation | primer |
| GACTCCTGGCCTTGACTTGA | forward | AdIYEx1 |
| TCTCTGTGGTGCTGATCCTG | forward | cf1 |
| GGAGGAGCAAAAACAAGAAGAGA | forward | cf1-117 |
| ACTGATGAGCACGGGAACC | forward | cf1-205 |
| TCCATCCTGAAAGTGCCTG | forward | C17c |
| ACATGTATACATGCTGCCAA | forward | C18 |
| CAGCGAAGGAAAGCACATTT | forward | AdIYEx5 |
| GGCGACCTGAAGGGGACT | forward | cf1-1915 |
| CTGTCCAGTCCTCAGGAAGC | forward | C21 |
| GAAGCATCCACCAAAGCG | forward | cf1-4679 |
| ACAGCGGGCGCTATGAGT | forward | cf1-4a |
| CAGGATCAGCACCACAGAGA | reverse | AdIYEx2 |
| CTGGGGAAGTTGGATTTTCTC | reverse | C17b |
| ACCAGGTTCCCGTGCTCA | reverse | cf1-227 |
| GCAAGAATCTGGGCTCTCAC | reverse | cf1 |
| ACTGTGATTCCCACCGTGAT | reverse | C17c |
| TTGTTTTGAGGAACGCCTCT | reverse | C18 |
| GGATGTGGGATCTGGTGAG | reverse | cf1-2079 |
| GGGTGTAATTTTCTCCCATTG | reverse | AdIYEx5 |
| CGTCCGTTTCAGCAGTGACA | reverse | cf1-4810 |
| CTGACGTCCGTCCTCTGC | reverse | cf1-4b |
| ATGGACAGTGATCCGGTTTC | reverse | cf1-6453 |
| TGAGCTGCACGATCAACCTC | reverse | cf1-6559 |

TABLE 7C
RT-PCR primer sequences for ADLY

| | | | | |
|---|---|---|---|---|
| | | Pos. in | Pos. in | ADL |
| Primer | Sequence (5′→3′) | ADLY¹ | ADLX | exon² |

Forward primer

| | | | | |
|---|---|---|---|---|
| AdIYEx1 | GACTCCTGGCCTTGACTTGA | 44-63 | — | 1 |
| cf1 | TCTCTGTGGTGCTGATCCTG | 184-203 | 184-203 | 2 |
| Ad1YEx5 | CAGCGAAGGAAAGCACATTT | 2177-2196 | — | 5 |
| C21 | CTGTCCAGTCCTCAGGAAGC | 5089-5108 | 5620-5639 | 5 |
| cf1-4a | ACAGCGGGCGCTATGAGT | 5971-5988 | 6502-6519 | 6 |

Reverse Primer

| | | | | |
|---|---|---|---|---|
| AdIYEx2 | CAGGATCAGCACCACAGAGA | 203-184 | 203-184 | 2 |
| cf1 | GCAAGAATCTGGGCTCTCAC | 914-895 | 1435-1416 | 5 |
| Ad1YEx5 | GGGTGTAATTTTCTCCCATTG | 3103-3083 | — | 5 |
| cf1-4b | CTGACGTCCGTCCTCTGC | 6143-6126 | 6631-6614 | 6 |
| cf1-6453 | ATGGACAGTGATCCGGTTTC | 7158-7139 | 7649-7630 | 7 |

¹ADLY refers to the gene predicted according to homology comparison with functional X-adlican.
²Numbering of exons is based on the exon/intron organization of the X-copy. Please note: RT-PCR with cf1for/rev would generate different-sized products from adlican copies. cf1-4a/cf1-6453 and C21/Cf1-4b amplification products encompass chromosome-specific restriction sites (cf1-4a/cf1-6453: Y-BamHI, X-PsyI; C21/cf1-4b: Y-NlaIII, X-SacI).

TABLE 8
RT-PCR primer sequences for exon trap clones

| Exon | | |
|---|---|---|
| trap | | |
| clone | Forward primer | Reverse primer |
| eta2 | GCACCATTAGTGCGCTTGT | GAGCATCAGGGGTGTCTTCT |
| eta3 | a: TTACATAGAATGGTAACTCCTTTTGC | |
| | b: AACTCCTTTTGCACCTCGTG | |
| eta4 | | a: GCTGATGCTCAGAGTGTGGA |
| | | b: GATTGCTGGCTGTGTCACC |
| etc1 | a: TTTAAAATTCCTTCTAAACTTTTTCC | |
| | b: CCCATTTCTGCTCAATTTTCA | |
| etc2 | a: GCTGAACATTATTTCTTTATTCCAGA | |
| | b | AGAGGACTAGGATTCATGGGATT |
| etc3 | | a: TGAAATGGCAAACCTTTCAGA |
| | | b: GGCAGAGACTCTCCTACATATTC |
| etc4 | TGGCCTATAAAGGGATCAATG | GGTGCAGGAGGGTGATTAAG |
| etc5 | a: GAAAGCCACCAAGAGTGGAC | |
| | b: ACCAATATCCAAGGGACATGA | |

The product size of eta2 is 175 bp and of etc4 166bp. For single exon-trap clones semi-nested PCR was carried out: a reflects the outer primer, b the inner one.

TABLE 9a
Homology comparison of exons

| | |
|---|---|
| Size (bp) | Nucleotide sequence |

| Exon | ADLX | ADLY | homology (%) |
|---|---|---|---|
| 1 | 127 | 127 | 85 |
| 2 | 215 | 217 | 97 |
| 3 | 129 | deleted | — |
| 4 | 390 | deleted | — |
| 5 | 4967 | 4958 | 93 |
| 6 | 900 | 944 | 95 |
| 7 | 3061 | 3097 | 94 |

TABLE 9b
Exon/intron boundaries of conserved exons

| | Intron/Exon | | Exon/Intron | |
|---|---|---|---|---|
| Exon | ADLX | ADLY | ADLX | ADLY |

| | | | | |
|---|---|---|---|---|
| 1 | GAGCTGCCTC | GAGCTGCCTC | CCAAGGACAGgtgaggaccc | CCAAGGATAGgtgaggaccc |
| 2 | tctacctcagGTATCCGAGA | tctacctcagGTATCCGAGA | TCAATTTGGGgtttgtacca | TCAATTTGGGgtttgtacca |
| 5 | tttgttttagGAATTCTGAA | tttgttttagGAATTCTGAA | GTTTCCACAGgtaatatgtt | GTTTCCACATgtaagatttt |
| 6 | ttttctccagGAGCTCTTAT | ttttctccagGAGTTCTTAT | CGCTCTTCAGgtaggcagct | CGCTTTTCAGgtaggcagct |
| 7 | ttttctgtagTTTTGATAGC | ttttctgtagTTTTGATAGT | ATATTCTCCCC | ATATTCTCCCC |

TABLE 10
Sequence divergence of genes/pseudogenes from the GCY region and their homologues

| | | | | DNA | Protein | Sequence |
|---|---|---|---|---|---|---|
| Gene pair | K₅ | K₆ | K₅/K₆ | divergence | divergence | compared (nt) |
| | Genes in GCY region | | | | | |
| | X/Y gene pairs | | | | | |
| ADLX/ADLY | 0.10 | 0.07 | 1.4 | 8 | 15 | 1260 |
| ARSF/ARSFP | 0.09 | 0.08 | 1.1 | 9 | 18 | 456 |
| RPS24PX/RPS24P1 | 0.16 | 0.09 | 1.8 | 11 | 22 | 357 |
| RPS24/RPS24P1* | 0.28 | 0.17 | 1.6 | 20 | 30 | 369 |
| ASSP4/ASSP6 | 0.10 | 0.08 | 1.3 | 9 | 20 | 1230 |
| ASS/ASSP6* | 0.17 | 0.09 | 1.9 | 11 | 22 | 1230 |
| | 1/Y gene pairs | | | | | |
| KIAA1470P1/KIAA1470PY | 0.05 | 0.03 | 1.7 | 4 | 7 | 1194 |
| KIAA1470/KIAA1470PY* | 0.34 | 0.18 | 1.9 | 22 | 35 | 1203 |
| | X/Y gene pairs - Group 4 | | | | | |
| ARSE/ARSEP | 0.05 | 0.04 | 1.2 | 4 | 9 | 615 |
| | X/Y gene pairs - Group 3 | | | | | |
| DFFRX/DFFRY | 0.33 | 0.05 | 6.6 | 11 | 9 | 7671 |
| | X/Y gene pairs - Group 2 | | | | | |
| SMCX/SMCY | 0.52 | 0.08 | 6.5 | 17 | 15 | 4623 |
| | X/Y gene pairs - Group 1 | | | | | |
| RBMX/RBMY | 0.94 | 0.25 | 3.8 | 29 | 38 | 1188 |

*If chromosome X- or 1-derived copies of genes from the GCY region were not functional, Y-copies were additionally compared with their functional progenitors.

### REFERENCES

De Rosa M, De Brasi D, Zarrilli S, Paesano L, Pivonello R, D'Agostino A, Longobardi S, Merola B, Lupoli G, Ogata T, Lombardi G. Short stature and azoospermia in a patient with Y chromosome long arm deletion. J Endocrinol Invest 1997; 20:623-28.
Foresta C, Ferlin A, Garolla A, Moro E, Pistorello M, Barbaux S, Rossato M. High frequency of well-defined Y-chromosome deletions in idiopathic Sertoli cell-only syndrome. Hum Reprod 1998; 13:302-7.
Henegariu 0, Hirschmann P, Kilian K, Kirsch S, Lengauer C, Maiwald R, Mielke K, Vogt P. Rapid screening of the Y chromosome in idiopathic sterile men, diagnostic for deletions in AZF, a genetic factor expressed during spermatogenesis. Andrologia 1993; 26:97-06.
Jones M H, Khwaja O S A, Briggs H, Lambson B, Davey P M, Chalmers J, Zhou C-Y, Walker E M, Zhang Y, Todd C, Ferguson-Smith M A, Affara N A. A set of ninety-seven overlapping yeast artificial chromosome clones spanning the human Y chromosome euchromatin Genomics 1994; 24:266-75.
Kirsch S, Weiβ B, De Rosa M, Ogata T, Lombardi G, Rappold G A. FISH deletion mapping defines a single location for the Y-chromosome stature gene, GCY. J Med Genet 2000; 37:593-9.
Kleirman S E, Yogev L, Gamzu R, Hauser R, Botchan A, Lessing J B, Paz G, Yavetz H. Genetic evaluation of infertile men Hum Reprod 1999; 14:33-38.
Lichter P, Cremer T. Human Cytogenetics: A Practical Approach. Oxford/New York/Tokyo: Oxford University Press, IRL 1992. Ma K, Inglis J D, Sharkey A, Bickmore W A, Hill R E, Prosser E J, Speed R M, Thomson E J, Jobling M Taylor K, Wolfe J, Cooke H J, Hargreave T B, Chandley A C. A Y chromosome gene family with RNA-binding protein homology: candidates for the azoospermia factor AZF controlling human spermatogenesis. Cell 1993; 31:1287-95.
Nelson D L, Ballabio A, Victoria M F, Pieretti M, Bies R D, Gibbs R A, Maley J A. Chinault A C, Webster T D, Caskey C T. Alu-primed polymerase chain reaction for regional assignment of 110 yeast artificial chromosome clones with a disease locus. Proc Natl Acad Sci USA 1991; 88:6157-61.
Ogata T, Matsuo N. Comparison of adult height between patients with XX and XY gonadal dysgenesis: support for a Y specific growth gene(s). J Med Genet 1992; 29:539-41.
Ogata T, Tomita K, Hida A, Matsuo N, Nakahori Y, Nakagome Y. Chromosomal localisation of a Y specific growth gene(s). J Med Genet 1995; 32:572-5.
Pryor J L, Kent-First M, Muallem A, Van Bergen A H, Nolten W E, Meisner L, Roberts K P. Microdeletions in the Y chromosome of infertile men. N Engl J Med 1997; 336:534-9.
Rao E, Weiss B, Fukami M, Rump A, Niesler B, Mertz A, Muroya K, Binder G, Kirsch S, Winkelmann M, Nordsiek G, Heinrich U, Breuning M H, Ranke M B, Rosenthal A, Ogata T; Rappold G A. Pseudoautosomal deletions encompassing a novel homeobox gene cause growth failure in idiopathic short stature and Turner syndrome. Nat Genet 1997; 16:54-63.
Reijo R, Lee T Y, Salo P, Alagappan R, Brown L G, Rosenberg M, Rozen S, Jaffe T, Straus D, Hovatta O, de la Chapelle A, Silber S, Page D C Diverse spermatogenic defects in humans caused by Y chromosome deletions encompassing a novel RNA-binding protein gene. Nat Genet 1995; 10:383-93.
Ried T, Baldini A, Rand T C, Ward D C. Simultaneous visualization of seven different DNA probes by in situ hybridization using combinatorial fluorescence and digital imaging microscopy. Proc Natl Acad Sci USA 1992; 89:1388-92.
Rousseaux-Prevost R, Rigot J-M, Delobel B, Lesur P, Collier F, Croquette M-F, Gauthier A, Mazeman E, Rousseaux J. Molecular mapping of a Yq deletion in a patient with normal stature. Hum Genet 1996; 98:505-7.
Salo P, K\ddot{aa}riäinen H, Page D C, de la Chapelle A. Deletion mapping of stature determinants on the long arm of the Y chromosome. Hum Genet 1995; 95:283-6.
Sargent C A, Boucher C A, Kirsch S, Brown G, Weiss B, Trundley A, Burgoyne P, Saut N, Durand C, Levy N, Terriou P, Hargreave T, Cooke H, Mitchell M, Rappold G A, Affara N A. The critical region of overlap defining the AZFa male infertility interval of proximal Yq contains three transcribed sequences. J Med Genet 1999; 36:6707.
Skare J, Drwinga H, Wyandt H, van der Spek J, Troxler R, Milunsky A. Interstitial deletion involving most of Yq. Am J Med Genet 1990; 36:394-7.
Smith D W, Marokus R, Graham Jr J M. Tentative evidence of Y-linked statural gene(s). Clin Pediatr 1985; 24:189-92.
Tanner J M, Whitehouse R H, Takaishi M. Standards from birth to maturity for height, weight, height velocity, and weight velocity: British children 1965. Parts I and II. Arch Dis Child 1966; 41:454-471,613-635.
Aho M, Härk\ddot{o}nen K, Suikkari A M, Juvonen V, Anttila L, and Lähdetie J. Y-chromosomal microdeletions among infertile Finnish men. Acta Obstet Gynecol Scand 2001; 80:652-656
Amselem S, Duquesnoy P, Attree O, Novelli G, Bousnina S, Postel-Vinay M C, and Goossens M. Laron dwarfism and mutations of the growth hormone-receptor gene. N Engl J Med 1989; 321:989-995
Bor P, Hindkjaer J, Kolvraa S, and Ingerslev H J. Y-chromosome microdeletions and cytogenetic findings in unselected ICSI candidates at a Danish fertility clinic. J Assist Reprod Genet 2002; 19:224-231
Bühler E M. A synopsis of the human Y chromosome. Hum Genet 1980; 55:1451-75
Court Brown W M. Males with an XYY sex chromosome complement. J Med Genet 1968; 5:341-359 Dattani M T, Martinez-Barbera J P, Thomas P Q, Brickman J M, Gupta R, Martensson I L, Toresson H, Fox M, Wales K J, Hindmarsh P C, Krauss S, Beddington R S, and Robinson I C. Nat Genet 1998; 19:125-133
de la Chapelle A. Nature and origin of males with XX sex chromosomes. Am J Hum Genet 1972; 24:71-105
Dillon N and Festenstein R Unravelling heterochromatin: competition between positive and negative factors regulates accessibility. TIG 2002; 18:252-258
Falkner F, Tanner J M (eds) Human growth 1985 2nd ed, Vol 3. Plenum Press, New York, N.Y.
¹Foresta C, Moro E, and Ferlin A. Y chromosome microdeletions and alterations of spermatogenesis. Endocr Rev 2001; 22:226-239
²Foresta C, Bettella A, Moro E, Roverato A, Merico M, and Ferlin A. Sertoli cell function in infertile patients with and without microdeletions of the azoospermia factors on the Y chromosome long arm. J Clin Endocrinol Metab 2001; 86:2414-2419
Friel A, Houghton J A, Maher M, Smith T, Noël S, Nolan A, Egan D, and Glennon M. Molecular detection of Y chromosome microdeletions: an Irish study. Int J Androl 2001; 24:31-36
Fujisawa M, Shirakawa T, Kanzaki M, Okada H, Arakawa S, and Kamidono S. Y-chromosome microdeletion and phenotype in cytogenetically normal men with idiopathic azoospermia. Fertil Steril 2001; 76:491-495
Ioulianos A, Sismai C, Fourouclas N, Patroclou T, Sergiou C, and Patsalis P C. A nation-based population screening for azoospermia factor deletions in Greek-Cypriot patients with severe spermatogenic failure and normal fertile controls, using a specific study and experimental design. Int J Androl 2002; 25:153-158
Kamp C, Huellen K, Fernandes S, Sousa M, Schlegel P N, Mielnik A, Kleiman S, Yavetz H, Krause W, Küpker W, Johannisson R, Schulze W, Weidner W, Barros A, and Vogt P H. High deletion frequency of the complete AZFa sequence in men with Sertoli-cell-only syndrome. Mol Hum Reprod 2001; 7:987-994
Kirsch S, Weiβ B, De Rosa M, Ogata T, Lombardi G, and Rappold G A. FISH deletion mapping defines a single location for the Y-chromosome stature gene, GCY. J Med Genet 2000; 37:593-599.
Kirsch S, Weiss B, Kleiman S, Roberts K, Pryor J, Milunsky A, Ferlin A, Foresta C, Matthijs G, and Rappold G A. The Y-chromosomal stature gene resides in close proximity to the centromere. J Med Genet 2002; 39:507-513.
Kuroda-Kawaguchi T, Skaletsky H, Brown L G, Minx P J, Cordum H S, Waterston R H, Wilson R K, Silber S, Oates R, Rozen S, and Page D C. The AZFc region of the Y chromosome features massive palindromes and uniform recurrent deletions in infertile men. Nat Genet 2001; 29:279-286.
Lahn B T, Page D C. Four evolutionary strata on the human X chromosome. Science 1999; 286:964-967
Li W H. Unbiased estimation of the rates of synonymous and nonsynonymous substitution. J Mol Evol 1993; 36:96-99
Machinis K, Pantel J, Netchine I, Leger J, Camand O J, Sobrier M L, Dastot-Le Moal F, Duquesnoy P, Abitbol M, Czernichow P, and Amselem S. Syndromic short stature in patients with a germline mutation in the LIM homeobox LHX4. Am J Hum Genet 2001; 69:961-968
Madgar L, Green L, Kent-First M, Weissenberg R, Gershoni-Baruch R, Goldman B, and Friedman E. Genotyping of Israeli infertile men with idiopathic oligozoospermia Clin Genet 2002; 62:203-207
Martinez M C, Bemabé M J, Gómez E, Ballesteros A, Landeras J, Glover G, Gíl-Salom M, Remohí J, and Pellicer A. Screening for AZF deletion in a large series of severely impaired spermatogenesis patients. J Androl 2000; 21:651-655
Maurer B and Simoni M. Y chromosome microdeletion screening in infertile men. J Endocrinol Invest 2000; 23:664670
Maurer B. Gromoll J, Simoni M, and Nieschlag E. Prevalence of Y chromosome microdeletions in infertile men who consulted a tertiary care medical centre: the Münster experience. Andrologia 2001; 33:27-33
Nakashima M, Koh E, Namiki M, and Yoshida A. Multiplex sequence-tagged site PCR for efficient screening of microdeletions in Y chromosome in infertile males with azoospermia or severe oligozoospermia Arch Androl 2002; 48:351-358
Netchine I, Sobrier M L, Krude H, Schnabel D, Maghnie M, Marcos E, Duriez B, Cacheux V, Moers A, Goossens M, Gruters A, and Amselem S. Mutations in LHX3 result in a new syndrome revealed by combined pituitary hormone deficiency. Nat Genet 2000; 25:182-186
Ogata T, Matsuo N. Comparison of adult height between patients with XX and XY gonadal dysgenesis: support for a Y specific growth gene(s). J Med Genet 1992; 29:539-541
Peterlin B, Kunej T, Sinkovec J, Gligorievska N, and Zorn B. Screening for Y chromosome microdeletions in 226 Slovenian subfertile men. Hum Reprod 2002; 17:17-24
Rao E, Weiss B, Fukami M, Rump A, Niesler B, Mertz-A, Muroya K, Binder G, Kirsch S, Winkelmann M, Nordsiek G, Heinrich U, Breuning M H, Ranke M B, Rosenthal A, Ogata T, and Rappold G A. Pseudoautosomal deletions encompassing a novel homeobox gene cause growth failure in idiopathic short stature and Turner syndrome. Nat Genet 1997; 16:54-63
Reijo R, Lee T Y, Salo P, Alagappan R, Brown L G, Rosenberg M, Rozen S, Jaffe T, Straus D, Hovatta O, de la Chapelle A, Silber S, and Page D C. Diverse spermatogenic defects in humans caused by Y chromosome deletions encompassing a novel RNA-binding protein gene. Nat Genet 1995; 10:383-393
Rogic S, Mackworth A K, and Ouellette F B F. Evaluation of gene-finding programs on mammalian sequences. Genome Res 2001; 11:817-832
Salido E C, Yen P H, Koprivnikar K, Yu L C, and Shapiro L J. The human enamel protein gene amelogenin is expressed from both the X and the Y chromosomes. Am J Hum Genet 1992; 50:303-316 Santos F R, Pandya A, and Tyler-Smith C. Reliability of DNA-based sex tests. Nat Genet 1998; 18:103
Sargent C A, Boucher C A, Kirsch S, Brown G, Weiss B, Trundley A, Burgoyne P, Saut N, Durand C, Levy N, Terriou P, Hargreave T, Cooke H, Mitchell M, Rappold G A, and Affara N A. The critical region of overlap defining the AZFa male infertility interval of proximal Yq contains three transcribed sequences. J Med Genet 1999; 36:670-677
Schiebel K, Winkelmann M, Mertz A, Xu X, Page D C, Weil D, Petit C, and Rappold G A. Abnormal XY interchange between a novel isolated protein kinase gene, PRKY, and its homologue, PRKX, accounts for one third of all (Y+)XX males and (Y−)XY females. Hum Mol Genet 1997; 6:1985-1989
Schwartz S, Zhang Z, Frazer K A, Smit A, Riemer C, Bouck J, Gibbs R, Hardison R, and Miller W. PipMaker—A web server for aligning two genomic DNA sequences. Genome Res 10; 577-586
Singh L, Jones K W. The use of heparin as a simple cost effective means of controlling background in nucleic acid hybridization procedures. Nucl Acids Res 1984; 12:627-5638
Smith D W, Marokus R, and Graham Jr J M. Tentative evidence of Y-linked statural gene(s). Clin Pediatr 1985; 24:189-192
Takahashi Y, Kaji H, Okimura Y, Goji K, Abe H, and Chihara K. Short stature caused by a mutant growth hormone. N Engl J Med 1996; 334:432-436
Tatsumi K. Miyai K, Notomi T, Kaibe K. Amino N, Mizuno Y, and Kohno H. Cretinism with combined hormone deficiency caused by a mutation in the PIT1 gene. Nat Genet 1992; 1:56-58
Tse J Y, Yeung W S, Ng E H, Cheng L N, Zhu H B, Teng X M, Liu Y K, and Ho P C. A comparative study of Y chromosome microdeletions in infertile males from two Chinese populations. J Assist Reprod Genet 2002; 19:376-383
Wajnrajch M P, Gertner J M, Harbison M D, Chua S C Jr, and Leibel R L. Nonsense mutation in the human growth hormone-releasing hormone receptor causes growth failure analogous to the little (lit) mouse. Nat Genet 1996; 12:88-90
Wimmer R, Kirsch S, Rappold G A, and Schempp W. Direct evidence for the Homo-Pan clade. Chrom Res 2002.10:55-61
Wu W, Cogan J D, Pfaffle R W, Dasen J S, Frisch H, O'Connell S M. Flynn S E, Brown M R, Mullis P E. Parks J S, Phillips J A 3rd, and Rosenfeld M G. Mutations in PROP1 cause familial combined pituitary hormone deficiency. Nat Genet 1998; 18:147-149
Yao G, Chen G, and Pan T. Study of microdeletions in the Y chromosome of infertile men with idiopathic oligo- or azoospermia. J Assist Reprod Genet 2001; 18:612-616

1. An isolated region of the Y chromosome between SKY1 and sY83 which encompasses the Y-specific growth gene GCY.

2. An isolated region according to claim 1 which is about 700 Kb in size.

3. An isolated region according to claim 1 in which the Y chromosome is a human chromosome.

4. An isolated region according to claim 1 which is between SKY8 and sY83.

5. An isolated region according to claim 1 which is between sY79 and sY81.

6. An isolated GCY protein, encoded by a region of the Y chromosome within the interval SKY1 and sY83.

7. An isolated GCY protein according to claim 6 encoded by a region within the interval SKY8 and sY83.

8. An isolated GCY protein according to claim 7 encoded by a region between sY79 and sY81.

9. An isolated GCY protein according to claim 8, which is ADLY or a functional fragment thereof.

10. A nucleic acid primer having a nucleic acid sequence selected from a nucleic acid sequence as recited in SEQ ID NOs: 1-26, SEQ ID NOs: 27-60, SEQ ID NOs: 61-71, SEQ ID NOs: 72-98, SEQ ID NOs: 99-121, SEQ ID NOs: 122-131, or SEQ ID NOs: 132-147.

11. A method of studying GCY localisation or identifying a GCY gene associated with height comprising the use of a primer according to claim 10 to selectively amplify or detect a region of a nucleic acid molecule.

12. An isolated protein having greater than 65% homology to a GCY protein encoded by a region of the Y chromosome within the interval SKY1 and sY83, and which contributes to the sex-related height difference in humans.

13. (canceled)
